(19) United States
(12) Patent Application Publication
(10) Pub. No.: US 2022/0017918 A1
(43) Pub. Date: Jan. 20, 2022

(54) Synthesis of Non-Native Proteins in Bombyx Mori by Modifying Sericin Expression

(71) Applicant: Kraig Biocraft Laboratories, Inc., Ann Arbor, MI (US)

(72) Inventors: Kimberly Kraig Thompson, Lansing, MI (US); Trevor Logan Kane, Dewitt, MI (US); Ana Laura Licon, Brighton, MI (US); Jimi Craig Miller, Williamston, MI (US)

(21) Appl. No.: 17/377,318

(22) Filed: Jul. 15, 2021

Related U.S. Application Data
(60) Provisional application No. 63/053,491, filed on Jul. 17, 2020.

(51) Int. Cl. C12N 15/85; A01K 67/04; C07K 14/47; C07K 14/435; C12N 15/11

(52) U.S. Cl. CPC C12N 15/8509; A01K 67/04; C07K 14/4741; C12N 2015/8518; C12N 15/11; A01K 2227/706; A01K 2217/054; C07K 14/43518

(57) ABSTRACT

Described herein are methods of producing transgenic Bombyx mori by targeting and modifying genomic regions associated with sericin proteins. Embodiments include vectors utilized for modifying one or more sericin genes. Embodiments include plasmid constructs utilized for molecular cloning of donor sequences configured for replacement of or insertion into a targeted sericin gene and utilized for transfection of Bombyx mori with the donor sequences. Embodiments include transgenic Bombyx mori that have been transfected with the donor sequences and are capable of producing a non-native protein product with minimized or prevented production of sericin.

### CROSS-REFERENCE TO RELATED APPLICATIONS

This application claims priority to and the benefit of U.S. Provisional Patent Application Ser. No. 63/053,491, filed Jul. 17, 2020 and titled “Method of Producing Non-Native Proteins in Bombyx Mori”, the entirety of which is incorporated herein by this reference.

### INCORPORATION BY REFERENCE OF SEQUENCE LISTING

The document filed in conjunction with this application includes a numerical listing of sequences corresponding to the sequences described herein, each identified by a unique SEQ ID NO. The 21824-8-1-SeqList.txt file was created on Jul. 15, 2021 and has a size of 126,730 bytes. The 21824-8-1-SeqList.txt file is expressly incorporated herein by this reference.

### BACKGROUND

### Technical Field

This disclosure generally relates to methods of producing transgenic Bombyx mori (i.e., domestic silkworm) by targeting and modifying genomic regions associated with the sericin protein.

### Related Technology

Bombyx mori is an insect from the Bombycidae moth family, most commonly referred to simply as the silkworm (in the larvae stage) or silk moth (in the adult stage). Bombyx mori were domesticated thousands of years ago in China for their ability to produce relatively large quantities of silk. Selective breeding has, over time, enabled domestic Bombyx mori to produce almost 10 times as much silk as their wild counterparts.

After a silkworm has molted four times, it will enter the pupal stage by forming a cocoon made of raw silk. The cocoon is typically formed from a single filament that can average more than 900 meters in length. The silk is harvested by steaming or boiling the cocoon before the adult moth can form and release protease enzymes, which would damage the silk of the cocoon.

Bombyx mori silk is made up of two major components: fibroin and sericin. Fibroin is produced in heavy chain, light chain, and glycoprotein P25 forms. When the silkworm produces silk, the heavy and light chains are linked by disulphide bonding, and the P25 integrates via non-covalent interactions. The sericin proteins are hydro-soluble and function to coat and adhere separate fibroin filaments as the silkworm generates the silk. Sericin proteins are glue-like proteins that coat the heavy chain and light chain proteins and allows neighboring silk threads to adhere together.

In commercial silk production, sericin proteins are regarded as unimportant byproducts and are typically removed. During production, cocoons are first boiled in an aqueous solution. This boiling results in the dissolving and removal of the sericin proteins, and the solution containing the sericin proteins is discarded. The cocoon threads are typically then woven into a textile product.

There have been attempts to produce transgenic silkworms capable of expressing non-native proteins, particularly spider silk proteins. However, thus far it has been challenging to transgenically produce spider silk with the desired mechanical characteristics, at appropriate scale, and in a cost-effective manner. Other protein production systems such as those that utilize microbes (e.g., E. coli or yeast-based systems) are limited by the inability of the microbes to handle large proteins and/or proteins associated with repetitive DNA sequences. Examples of such proteins include spider silk proteins, collagen, elastin, and other fibrous proteins. Accordingly, there are a number of disadvantages with conventional protein production technology.

### SUMMARY

As discussed above, it has been challenging to produce spider silk at large scale and in a cost-effective manner in part due to the inability to culture spiders en masse for this purpose. Moreover, although there have been attempts to utilize other organisms to produce spider silk and other large, industrially relevant proteins, these efforts have also met significant challenges such as the inability of microbial systems to generate large proteins that include several repeating structural motifs, the need to purify the intended product, and/or difficulty in achieving an end product with the desired mechanical properties.

Silkworms transfected with spider silk DNA or DNA encoding other proteins of interest is one promising approach to achieving effective and economical production of enhanced protein products. Silkworms have the inherent ability to spin fibers at relatively high purity levels, reducing the need for complicated downstream processing of the product. Silkworms have also been cultured for thousands of years, and a mature sericulture industry is already in place.

Embodiments disclosed herein are directed to methods of genetically knocking out one or more of the sericin genes Ser1, Ser2, and/or Ser3 (or portions thereof). Certain embodiments disclosed herein are directed to vectors with donor sequences that encode non-native proteins for replacing all or a portion of the native sericin gene(s). In some embodiments, the non-native proteins are not incorporated into the silk fibroin of the silkworm but are instead separated from the rest of the cocoon during normal silk processing and then purified. Embodiments can therefore beneficially leverage the normal Bombyx mori silk production process to generate one or more useful proteins instead of what is normally considered a waste product.

Certain embodiments disclosed herein include plasmid constructs for molecular cloning of donor sequences configured for replacement of one or more of the sericin genes (or portions thereof), and/or for transfection of Bombyx mori with the donor sequences. Certain embodiments disclosed herein are directed to transgenic Bombyx mori that have been transfected with such donor sequences and that can produce one or more non-native proteins (e.g., fibrous proteins such as spider silk, collagen, elastin, keratin, fibrin, or other medically and/or industrially relevant proteins). Certain embodiments disclosed herein are directed to the protein product produced by such transgenic Bombyx mori.

In some embodiments, the donor sequences and associated vectors are larger and more complex than those utilized in the prior art. Their successful use has beneficially led to transgenic Bombyx mori capable of producing a protein product with a high proportion of non-native protein, with little to no negative effects on the overall production of protein from the silkworms. In certain embodiments where the result is a fibrous product, the resulting product can provide enhanced mechanical properties such as enhanced strength and elasticity.

Certain embodiments increase the efficiency of silkworm protein production systems via modification of one or more sericin genes. For example, certain embodiments modify one or more sericin genes to limit or prevent sericin production and thereby limit the amount of energy and resources wasted by the silkworms in producing the associated sericin proteins. Reducing or preventing sericin production also simplifies processing of the protein product generated by the silkworms. Certain embodiments further make use of the inherent capabilities of silkworms by replacing all or part of one or more sericin genes with one or more donor sequences encoding for non-native proteins of interest. Such embodiments not only limit the generation of waste products, but instead direct the silkworms toward generation of beneficial, medically and/or industrially relevant proteins.

In one embodiment, a method of producing transgenic Bombyx mori comprises: providing a gene editing assembly that includes a nuclease configured to target one or more locations within a sericin gene of the Bombyx mori; providing a vector having a donor sequence that encodes a non-native protein; and using the gene editing assembly to incorporate the vector into one or more Bombyx mori cells. The vector may be configured to target the Ser1 gene, Ser2 gene, or Ser3 gene. Additional vectors may be included/utilized to target more than one region within a particular sericin gene and/or to target more than one sericin gene. For example, a set of vectors may be provided that is configured to target one or more portions of Ser1 and one or more portions of Ser2, or to target one or more portions of Ser1 and one or more portions of Ser3, or to target one or more portions of Ser2 and one or more portions of Ser3, or to target one or more portions of Ser1, one or more portions of Ser2, and one or more portions of Ser3.

The method may be carried out using a gene editing assembly such as Mad7 or CRISPR Cas9. The donor sequence may comprises a sequence that encodes for a spider silk protein, such as an A2S8 protein, a MaSp1 protein, a MaSp4 protein, or a combination thereof. The donor sequence comprises a sequence that encodes for a scleroprotein, such as a collagen, elastin, keratin, or fibrin. The scleroprotein may be a human protein.

Certain embodiments are directed to a transgenic Bombyx mori silkworm made according to the methods described herein. Certain embodiments are directed to a protein made by such transgenic Bombyx mori.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various FIGURES, and the various elements depicted are not necessarily drawn to scale, wherein:

FIG. 1 illustrates an exemplary plasmid map showing, in this example, a collagen donor sequence designed for introduction into the Bombyx mori, the vector portion of the plasmid also including homologous arms designed to enable knock-in of the donor sequence following knockout of all or a portion of the Ser2 gene.

### DETAILED DESCRIPTION

Overview of Transgenic Bombyx mori

Bombyx mori silk is made up of two major components: fibroin and sericin. Fibroin is produced in heavy chain, light chain, and glycoprotein P25 form. When the silkworm produces silk, the heavy and light chains are linked by disulphide bonding, and the P25 integrates via non-covalent interactions. The sericin proteins are hydro-soluble and function to coat and adhere separate fibroin filaments as the silkworm generates the silk. In commercial silk production, the sericin is typically removed as an unimportant side product.

As discussed above, sericin proteins are seen as waste products and are typically discarded as part of the aqueous solution following boiling of the silkworm cocoons. At present, three primary sericin genes have been discovered: sericin1, sericin2, and sericin3. Some reports have also described a fourth sericin gene: sericin4. All the sericin genes are located on chromosome 11. Embodiments described herein are primarily directed to modification of sericin1 (Ser1), sericin2 (Ser2), and sericin3 (Ser3), although similar approaches for modifying sericin4 (Ser4) and/or other sericin genes discovered in the future will be readily understood by the skilled person in light of this disclosure.

This disclosure presents embodiments that increase the efficiency of silkworm protein production systems via modification of one or more sericin genes. For example, certain embodiments modify one or more sericin genes to limit or prevent sericin production and thereby limit the amount of energy and resources wasted by the silkworms in producing the associated sericin proteins. Reducing or preventing sericin production also simplifies processing of the protein product generated by the silkworms. Certain embodiments further make use of the inherent capabilities of silkworms by replacing all or part of one or more sericin genes with one or more donor sequences encoding for non-native proteins of interest. Such embodiments not only limit the generation of waste products, but instead direct the silkworms toward generation of beneficial, industrially relevant proteins.

As used herein, “modification” of one or more sericin genes includes embodiments where an entire sericin gene, or one or more portions thereof, are knocked out of the silkworm genome and replaced with a knock-in insert (e.g., using a truncation vector). In some knockout embodiments, at least about 50% of a sericin gene is knocked out, or at least about 60%, or at least about 70%, or at least about 80% of a sericin gene is knocked out. Although typically less preferred, the term also includes embodiments where one or more inserts are inserted at a position within a sericin gene and/or at a position functionally adjacent to the gene (e.g., using an insertion vector).

In some embodiments, a sericin gene is targeted in a manner that retains its native promoter within the genome. As a result, the resulting transgenic silkworm can utilize the native promoter for expression of the knocked-in insert. Alternative embodiments target the relevant promoter for inclusion in the knockout portion and include one or more separate promoter sequences as part of the knock-in insert.

An example of the Ser1 gene is provided as SEQ ID NO:1. An example of the Ser2 gene is provided as SEQ ID NO:2. An example of the Ser3 gene is provided as SEQ ID NO:3. Variations of these genes can occur due to differences in particular silkworm varieties, and the disclosed sequences are exemplary only. The skilled person will understand that the principles and components described herein can be utilized with other variations of sericin gene with only minor or no modification required.

Exemplary DNA targets associated with specific locations of the Ser1 gene for targeting by guide RNAs (gRNAs) are provided by SEQ ID NO:4 to SEQ ID NO:13. These sequences represent suitable portions of Ser1 to which gRNAs may target in a Mad7 system in which the PAM sequence is YTTN.

Exemplary DNA targets associated with specific locations of the Ser2 gene for targeting by gRNAs are provided by SEQ ID NO:14 to SEQ ID NO:23. These sequences represent suitable portions of Ser2 to which gRNAs may target in a Mad7 system in which the PAM sequence is YTTN.

Exemplary DNA targets associated with specific locations of the Ser3 gene for targeting by gRNAs are provided as SEQ ID NO:24 to SEQ ID NO:33. These sequences represent suitable portions of Ser3 to which gRNAs may target in a Mad7 system in which the PAM sequence is YTTN.

The locations of the corresponding sericin gene where these exemplary gRNAs target can be found by matching the gRNA target locations (or their reverse complements) to the corresponding location in the respective sericin gene (e.g., as provided by SEQ ID NO:1, SEQ ID NO:2, or SEQ ID NO:3). In some embodiments, reverse complements of one or more of the foregoing may additionally or alternatively be utilized as suitable gRNA targets.

The specific portions of a sericin gene targeted for knockout will vary somewhat depending on the particular gene editing process utilized. This is a result of inherent differences in gene editing techniques. For example, the standard Cas9 nuclease requires a protospacer adjacent motif (PAM) with sequence NGG, whereas the standard Mad7 endonuclease requires a PAM with sequence YTTN. Other Cas9 or Mad7 type nucleases, or other gene editing nucleases may have other associated PAM sequences, and thus the corresponding gRNAs may be varied accordingly. Other gene editing techniques such as those using transcription activator-like effector nucleases (TALENs) or zinc finger nucleases (ZFNs) have other inherent characteristics that must be accounted for when selecting a particular target site within the targeted sericin gene. Although specific examples described herein relate to a Mad7 gene editing process, the skilled person, in light of the teachings of this disclosure, is able to determine appropriate targets for such other gene editing processes.

### Exemplary Donor Inserts

As mentioned above, one or more sericin genes (or portions thereof) may be knocked out and replaced by donor sequences that encode for industrially and/or medically relevant proteins. Examples of such proteins include spider silk proteins, other fibrous proteins such as collagen, elastin, keratin, fibrin, or combinations thereof.

Many spider silks provide superior mechanical properties and would be beneficial for a variety of applications. However, cost-effective and appropriately scaled production of such silks has been elusive due to the technical challenges involved with producing the silk. The vectors and related methods described herein include spider silk protein sequences that enable the resulting transgenic silkworms to produce an enhanced silk product with beneficially enhanced mechanical properties.

Examples of sequences encoding spider silk proteins that can be included in the donor insert include those related to the proteins MaSp2, flagelliform, A2S8 (which includes alternating repeating motifs of MaSp2 and flagelliform), MaSp1, MaSp4, and MiSp. Although these proteins are presently preferred, other spider protein sequences may additionally or alternatively be included. Particularly preferred sequences are those associated with tangle-web weaver spiders (e.g., Latrodectus hesperus) and orb-weaver spiders such as golden orb-weaver spiders (Nephila) and the Darwin's bark spider (Caerostris danvini). These types of spiders can produce silk with extremely beneficial mechanical properties.

In some embodiments, a donor insert encodes a single protein. In other embodiments, the donor insert includes sequences that encode two or more proteins. For example, a donor insert may include a set of sequences that encodes two or more spider silk proteins such as A2S8, MaSp1, and/or MaSp4. Donor inserts may additionally or alternatively include repeated sequences that would each separately encode the same protein. For example, a sequence that encodes for a particular protein may be repeated multiple times within the insert in order to provide a translated protein that is multiple times longer than the native protein, thereby providing desired differences in mechanical properties.

An example of an effective sequence that encodes for A2S8 is provided by SEQ ID NO:34. The A2S8 protein is a combination of alternating repeating motifs of MaSp2 and flagelliform that beneficially provides effective strength and elasticity.

Examples of effective sequences that encode for MaSp1 are provided by SEQ ID NO:35 (Caerostris darwini), SEQ ID NO:36 (Latrodectus hesperus), and SEQ ID NO:37 (Nephila clavipes). An Example of an effective sequence that encodes for MaSp4 is provided by SEQ ID NO:38 (Caerostris danvini).

In some embodiments, the donor insert can further include a 5′ homology arm and/or a 3′ homology arm to promote integration with the host genome.

An exemplary 5′ homology arm for a Ser1 vector is provided by SEQ ID NO:39. An exemplary 3′ homology arm for a Ser1 vector is provided by SEQ ID NO:40. An exemplary 5′ homology arm for a Ser2 vector is provided by SEQ ID NO:41. An exemplary 3′ homology arm for a Ser2 vector is provided by SEQ ID NO:42. An exemplary 5′ homology arm for a Ser3 vector is provided by SEQ ID NO:43. An exemplary 3′ homology arm for a Ser3 vector is provided by SEQ ID NO:44.

These homology arm sequences may be varied to some extent based on the particular Bombyx mori variants utilized, through modification of end portions that transition between the terminal domain sequences and the donor protein sequence(s), and/or through modification of end portions that transition between the terminal domain sequences and homologous arm sequences, for example.

In some embodiments, the donor DNA insert may additionally include sequences encoding for various tags and/or reporters. The spider silk may, for example, be fused with a reporter, such as luciferase, or with an N- or C-terminal epitope tag, such as FLAG, 6×-His, or other epitope tag known to those having skill in the art.

Although many of the above examples are directed to donor sequences encoding for spider silk, inserts encoding for one or more non-spider and/or non-silk proteins may additionally or alternatively be included in the vectors described herein. For example, some vectors may include donor sequences encoding for other types of fibrous proteins (i.e., scleroproteins) such as collagen, elastin, fibrin, various forms of keratin (e.g., in addition to or as an alternative to the spider silk proteins described herein), or combinations thereof. Vectors may also include donor sequences encoding for non-fibrous proteins such as proinsulin, human interferons, human growth hormones, human factor VIII, or any other medically or industrially useful protein.

In some embodiments, one or more of the donor sequences encode for human scleroproteins or other human proteins. Examples of donor sequences that encode for human scleroproteins include those that encode for a human collagen protein, a human elastin protein, and/or a human keratin protein.

Exemplary donor sequence encoding for human collagen proteins are provided by SEQ ID NO:45 (Homo sapiens COL1A1; collagen alpha 1 chain isoform X1) and SEQ ID NO:46 (Homo sapiens; collagen alpha 1 chain).

An exemplary donor sequence encoding for an elastin protein is provided by SEQ ID NO:47 (Homo sapiens ELN; precursor tropoelastin isoform variant 1).

An exemplary donor sequence encoding for a keratin protein is provided by SEQ ID NO:48 (Homo sapiens KRT16; Keratin, type I cytoskeletal 16).

An exemplary donor sequence encoding for Caddisfly (Trichoptera) silk is provided by SEQ ID NO:49 (Rhyacophila obliterate; RoHF).

### Gene Editing Methods

Various gene editing methods may be utilized to target and modify one or more sericin genes of Bombyx mori. Most gene editing methods rely on targeted endonuclease activity and vary based on the particular endonuclease utilized and the corresponding targeting technique inherent to the endonuclease used. ZFNs or TALENs may be utilized but are typically less preferred due to the necessity of designing and constructing new for each target. Presently, more preferred methods include those that utilize clustered regularly interspaced short palindromic repeats (CRISPR) methods, including those that make use of the Mad7 nuclease or the Cas9 nuclease, for example.

The choice of gene editing process utilized to form the transgenic Bombyx mori affects the design of gRNAs and/or the particular portion of the sericin gene targeted for nuclease activity. That is, particular portions of the sericin gene targeted for knockout and/or as an insertion site will vary somewhat depending on the particular gene editing process utilized due to inherent differences in the target requirements and activity of the different nucleases (e.g., different PAM sequence requirements).

Although the particular examples of gRNAs and vectors described herein are designed for Mad7 systems, other gRNAs and/or target sites may be utilized where other gene editing systems are used. Although the particular target site of the sericin gene may vary with different systems and/or with different gRNAs in order to accommodate different nuclease functionality, the target should preferably still be within about 200 base pairs, more preferably about 100 base pairs, even more preferably within about 50 base pairs, of the target site when the disclosed gRNAs and their corresponding nuclease systems are utilized.

### Vector Construction

Vectors utilized to modify one or more sericin genes include one or more spider silk sequences and/or other sequences encoding for proteins of interest, such as one or more of the sequences provided by SEQ ID NO:34 to SEQ ID NO:38 and SEQ ID NO:45 to SEQ ID NO:49. The vectors may also include homology arms such as provided by SEQ ID NO:39 to SEQ ID NO:44 as appropriate for the specific sericin gene targeted.

The vectors also include homologous arms designed to guide insertion of the donor sequence(s) into the targeted portion of the sericin gene. The form of the homologous arms will vary depending on the particular site of the sericin gene targeted for nuclease activity. The homologous arms are designed to have sufficient homology to the remaining upstream and downstream portions of the Bombyx mori genome following nuclease activity in order to guide appropriate insertion via homology directed repair. The exemplary homologous arm sequences disclosed herein may be varied somewhat to account for differences in sericin gene variants and/or different nuclease target sites as appropriate.

The use of a truncation vector (with minimal or no homology arms) versus an insertion vector involves different tradeoffs, and one may be preferred over another depending on particular application needs. For example, a truncation vector provides a resulting protein product with a higher proportion of non-native protein due to the removal of much of the native sericin sequences. On the other hand, an insertion vector adds to the overall size of the resulting proteins, which can beneficially affect mechanical properties of the silk in some situations.

The donor sequences described herein may be much larger than donor sequences utilized in prior Bombyx mori vectors. For example, a vector may include a donor sequence portion (i.e., the portion not including homologous arms) of greater than about 2 kbp, or greater than about 4 kbp, or greater than about 6 kbp, or greater than about 8 kbp, or greater than about 10 kbp, or greater than about 12 kbp, or greater than about 14 kbp, greater than about 16 kbp, or greater than about 18 kbp. A donor sequence may therefore range in size from about 2 kbp to about 20 kbp, though other ranges utilizing any two of the foregoing values as endpoints may also be utilized.

The large relative size of the donor sequence portion allows for a large resulting protein and the concomitant benefits to mechanical properties associated therewith. The size of the donor sequence portion may also be large relative to the homologous arms used to guide insertion, and yet still enable successful introduction into one or more of the sericin genes. The homology arms are typically about 500 bp to 1 kbp, for example. Typically, the donor sequence insert is approximately the same size as the homology arms. Here, however, the disclosed vectors proved effective even though the donor sequence portion can be more than 2 times the size of the average size of the homology arms. More typically, the donor sequence is more than 5 times, more than 8 times, more than 12 times, more than 16 times, more than 20 times, more than 24 times, more than 28 times, more than 32 times, more than 36 times, or more than 40 times the average size of the homology arms. A donor sequence may therefore be about 2 to about 40 times the average size of the corresponding homology arms. For example, an insert of about 20 kbp may be paired with homology arms of about 500 bp. Other ranges utilizing any two of the foregoing values as endpoints may also be utilized.

### Plasmid Construction & Cell Transfection

The vectors described herein may be inserted into plasmids. The plasmids may include features known in the art for enabling cloning and amplification. The plasmid may include an origin of replication, a suitable site for cloning (e.g., a multiple cloning site), a selection gene (e.g., ampicillin resistance), various regulatory sequences (e.g., promoters, binding sites, lac promoter and operon, etc.), and primer sites, for example. Various plasmid backbones are known in the art and are suitable for use with the vectors described herein. Examples include pUC57 and other plasmids of similar function and ability to receive vectors with the sizes disclosed herein.

In some embodiments, a plasmid can include the vector sequence as well as a sequence encoding for the nuclease (e.g., Cas9 or Mad7) of the associated gene editing process intended for incorporating the vector into one or more sericin genes. However, presently preferred embodiments deliver the nuclease and corresponding gRNAs separately in order to improve delivery and incorporation into the genome by preventing the plasmids from becoming too large.

Plasmids may be delivered to the target Bombyx mori cells via one or more suitable transfection methods known in the art. For example, silkworm eggs may be transfected via microinjection, electroporation, other transfection method, or combinations thereof. Following transfection, the plasmids may be linearized using targeted restriction enzymes and/or other known methods. In some embodiments, a nuclease target site associated with the nuclease of the corresponding gene editing method may be cloned into the plasmid such that the plasmid is itself targeted and linearized by the same nuclease used to target the host genome.

Introduction of the plasmids into the silkworm egg should preferably be done as soon as possible upon oviposition of the eggs. To aid in identifying possible transgenic eggs, a fluorescent marker such as GFP, dsRED, YFP or any other similar colorimetric protein can be linked to a silkworm egg stage promoter such as actin A3, Nos, 3xP3, or any other similar promoter. This promoter-colorimetric protein can then be integrated into a neutral site of the silkworm genome to ensure heritability. As described herein, incorporation to the neutral site can be done with any of the commonly used genetic engineering methods including but not limited to CRISPR, TALENs or Mad7.

In one embodiment, eggs that display the desired marker are then allowed to hatch, and silkworm from the GO generation are interbred. Eggs from the F1 generation are then screened once again via the appropriate fluorescent method. F1 eggs that have the fluorescent marker separated and tracked throughout their lifecycle. Molts from individual silkworms are collected, DNA is extracted, and genetic markers and/or insertions or deletions are screened for via PCR or other sequencing methods.

Protein samples can be extracted from the silkworm silk gland during 5th instar and subjected to mass spectrometry, western blotting, or other forms of protein verification. Upon establishment of a consistent protein of interest producing strain, scaled up protein production can begin. Protein production through silkworms can involve numerous approaches, including but not limited to allowing the silkworm to naturally secrete the protein during cocoon spinning steps or excising silk glands from the 5th instar silkworms. Following collection of the proteins, cocoons are dissolved according to best practices, and the proteins affinity purified. In the case of the silk gland extraction, the glands are dissolved, and proteins of interest are purified.

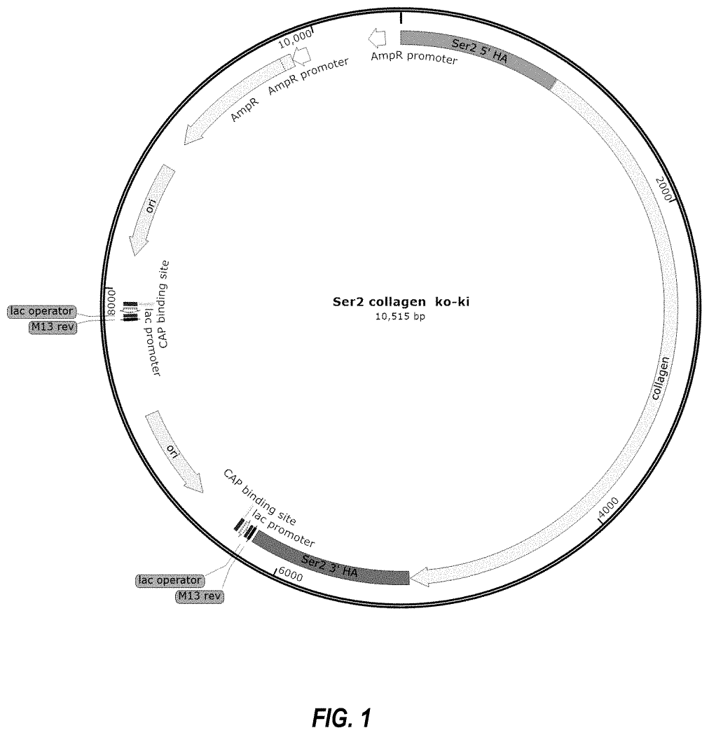

FIG. 1 illustrates an exemplary plasmid map showing a donor sequence (collagen in this example) designed for introduction into the Bombyx mori genome, the vector portion of the plasmid also including homologous arms designed to enable knock-in of the donor sequence following knockout of all or a portion of the Ser2 gene.

### Abbreviated List of Defined Terms

With respect to various terms of art and molecular biology details disclosed herein, reference is made to Sambrook, Fritsch, Maniatis, Molecular Cloning, A LABORATORY MANUAL (2d Edition, Cold Spring Harbor Laboratory Press, 1989) (especially Volume 3), and Kendrew, THE ENCYCLOPEDIA OF MOLECULAR BIOLOGY (Blackwell Science Ltd 1995). When combined with the teachings of this disclosure, the teachings of these references can be suitably modified, without undue experimentation, to enable the skilled artisan to utilize molecular biology techniques to construct the various vectors disclosed herein, to clone vectors into suitable plasmids, and to transfect and form recombinant organisms (e.g., E. coli, yeast, Bombyx mori) useful for generating high molecular weight proteins of interest.

As used herein, a “non-native” protein is a protein that is not natively produced by standard (not artificially genetically modified) forms of Bombyx mori. For example, spider proteins or human proteins are non-native with respect to a Bombyx mori protein production system.

As used herein, “modification” of one or more of the sericin genes includes embodiments where the entire gene, or one or more portions thereof, are knocked out of the silkworm genome and replaced with a knock-in insert (e.g., using a truncation vector). The term also includes embodiments where one or more inserts are inserted at a position within the gene and/or at a position functionally adjacent to the gene (e.g., using an insertion vector).

The terms “donor sequence”, “donor sequence portion”, “donor portion”, “donor insert”, and related terms are used herein to refer to the portion of a vector not including the homology arms intended to guide insertion of the vector to the target site within the target sericin gene. The donor sequence may include an N-terminal domain (NTD) and/or a C-terminal domain (CTD) sequence in addition to one or more protein encoding sequences. Alternatively, the donor sequence may omit the NTD and CTD sequences. The terms “vector”, “insertion vector”, and the like are used to refer to the full sequence that includes the donor sequence (or multiple combined donor sequences) and the upstream and downstream homology arms.

The terms “homologous arms” and “homology arms” are used interchangeably herein to refer to the portion of the vector intended to be homologous to a corresponding portion of the native gene on each side of the targeted location where introduction of the donor sequence is intended. Depending on where the sericin gene is targeted for nuclease activity, the NTD and CTD of the vector (if included) can act in whole or in part as homology arms.

It should be understood that the proteins and the nucleic acids encoding them may differ from the exact sequences illustrated and described herein. Thus, this disclosure includes related sequences with deletions, additions, truncations, and substitutions to the sequences shown, so long as the sequences function in accordance with the methods of the invention. Accordingly, nucleotide sequences encoding functionally equivalent variants of the illustrated sequences and proteins are included in this disclosure. For instance, changes in a DNA sequence that do not change the encoded amino acid sequence, as well as those that result in conservative substitutions of amino acid residues, one or a few amino acid deletions or additions, and/or substitution of amino acid residues by amino acid analogs are those which will not significantly affect properties of the encoded polypeptide.

Conservative amino acid substitutions include glycine/alanine; valine/isoleucine/leucine; asparagine/glutamine; aspartic acid/glutamic acid; serine/threonine/methionine; lysine/arginine; and phenylalanine/tyrosine/tryptophan. Amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of a leucine with an isoleucine or valine, or vice versa; an aspartate with a glutamate or vice versa; a threonine with a serine or vice versa; or a similar conservative replacement of an amino acid with a structurally related amino acid, will typically not have a major effect on activity and function of the overall protein. Proteins having substantially the same amino acid sequence as the sequences illustrated and described but possessing minor amino acid substitutions that do not substantially affect the activity or function of the protein are, therefore, within the scope of this disclosure.

Nucleotide sequences that have at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the disclosed sequences may be considered functional equivalents.

Sequence identity or homology may be determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877. Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use according to the present invention is the WU-BLAST (Washington University BLAST) version 2.0 software. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877).

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A method of producing transgenic Bombyx mori, the method comprising:
providing a gene editing assembly that includes a nuclease configured to target one or more locations within a sericin gene of the Bombyx mori;
providing a vector having a donor sequence that encodes a non-native protein; and
using the gene editing assembly to incorporate the vector into one or more Bombyx mori cells.

2. The method of claim 1, wherein the gene editing assembly is configured to target the Ser1 gene, and wherein the vector is configured to enable incorporation of the donor sequence into the Ser1 gene.

3. The method of claim 2, wherein the gene editing assembly includes one or more guide RNAs (gRNAs) for targeting the Ser1 gene, the one or more gRNAs configured to target one or more of SEQ ID NO:4 through SEQ ID NO:13.

4. The method of claim 1, wherein the gene editing assembly is configured to target the Ser2 gene, and wherein the vector is configured to enable incorporation of the donor sequence into the Ser2 gene.

5. The method of claim 4, wherein the gene editing assembly includes one or more gRNAs for targeting the Ser2 gene, the one or more gRNAs configured to target one or more of SEQ ID NO:14 through SEQ ID NO:23.

6. The method of claim 1, wherein the gene editing assembly is configured to target the Ser3 gene, and wherein the vector is configured to enable incorporation of the donor sequence into the Ser3 gene.

7. The method of claim 6, wherein the gene editing assembly includes one or more gRNAs for targeting the Ser3 gene, the one or more gRNAs configured to target one or more of SEQ ID NO:24 through SEQ ID NO:33.

8. The method of claim 1, wherein the gene editing assembly is a Mad7 assembly.

9. The method of claim 1, wherein the donor sequence comprises a sequence that encodes for an A2S8 protein, a MaSp1 protein, a MaSp4 protein, or a combination thereof.

10. The method of claim 9, wherein the donor sequence includes a sequence associated with an orb-weaver spider.

11. The method of claim 10, wherein the orb-weaver spider is Caerostris darwini.

12. The method of claim 1, wherein the donor sequence comprises a sequence that encodes for a scleroprotein.

13. The method of claim 12, wherein the donor sequence comprises a sequence that encodes for a collagen, elastin, keratin, or fibrin.

14. The method of claim 13, wherein the collagen, elastin, keratin, or fibrin is a human collagen, elastin, keratin, or fibrin.

15. The method of claim 1, wherein the collagen, elastin, keratin, or fibrin is a human collagen, elastin, keratin, or fibrin.

16. A transgenic Bombyx mori silkworm made according to the method of claim 1.

17. A protein made by the transgenic Bombyx mori silkworm of claim 16.

18. The protein of claim 17, wherein the protein includes spiker silk.

19. The protein of claim 18, wherein the spider silk comprises an A2S8 protein, a MaSp1 protein, a MaSp4 protein, or a combination thereof.

20. The protein of claim 17, wherein the protein includes a human scleroprotein.


SEQUENCE LISTING (49 sequences)

SEQ ID NO: 1 - DNA, 29965 bp, Bombyx mori
cttgatcaaacttcgttttcgtataaaacgcgttggcccaaccactttggcatagtcgtcttatcatcgggtctctaaggatcaagcgatccaaagaccgccaacatgcgtttcgttctgtgctgcactttgattgcgttggctgtgagtatcattgcttcgttatcaacaatgacgtatttactaagaacactcttagatatgccttcaaattaaagctttcaaagctctgaagttcaccaaatgcgactgttttagcgtaagctatttctatcccccaacagccatttagcgactacccgaaaatcactcgatttaacttgggagtttctgcaatttaaaagttcacaggtcgtctccgattatacttttaaacgcttcgcgaaaatatcagagttattaaaaagttgccacaaaatctttctgtccactaaggactggcgtttagcagcaaatcatctagattttgggtggttggttcagaattttccattttactcttgggagtcgtttcggtctgtattccctacgcattattagaatccatcgaatgtaatttgtataattacgagttaaatactgtgtacaaaccaatatgtaaatttttattcaactagtttattgtttttaacttataggtaccagttttgaaacgaacttcagagttatctagaaaaaacataattatgagtagctttactaatctatattaaattacgctttaatacttttaatagcttacttggggaaatagtgatttttcatgtaataacagtccacttggtcataagcagcttttatgggcttgcgctggatatgaatgccgcagaaccaacgatcgttggagtagacggggtcttgagtggagaccgcgtgccggcaaacgtagcgtgggacgcctcctgaccaagtggagggctggcttgcgatttggaagaggcctatgtctagcagggagtgattaggagctgatgattgtggtcataattgacagccacttcgtttatggcaatcaaatatcctgggcattttgagggtattttgctaaacgaagacattagaaggatgggcgagctcacagcccacctggtgttaagtggttactggagcccatagacatctacgacgtaaatgcgccacccaccttgagatataagttctaaggtctcagtatagttacaaccgctgccccgcccttcaaaccgaaacgcattaactgcttcacggcagaaataggcagggtggtggtacctacccgtgcggacccacaagaggtcctacctccagtccaatggtatcccaaaattttatacatataataataataataataataataatagcctttttattcctaaacatagggttacaagaggtccctaagttcggtgtgccttttggcataggcctcctccaattccttccaatgggtcctatcccttgctactctcagccagaatggtcccgctgtctgcttcaagtcatcttcccatctcctgatttgacgacccctacttcgtttgccgtctcttggttgccagagggtgacttgcttgctccatttgttaatggggtggcgtagcatatgcccagcccatctccatttttggacgtctatccgggtgagaatgtctgtgagtttcgttcttgttcttatgtctacgttcctaactctatctttgatttttaaccctagcatgcttctttccatggcacgttggcattttattagcttttccctgtggttaagtgtcattgaccaagtttcgcagccataagtaagaactggtagaatacaggtctcgaatgttttctttttgatgtgattgcctagatccttacttttcactatttccttcagtgaccagtattttctccaaccttgggctattcgcctggttatttccttggtcattacatctgaaggggatataatctggcctaagtaattatattctttgacatattcgatcttgatgccatctaccataacgtctatttcccttgagtttgtcatcaattttgtttttctgccattgatttccagaccagcttccctacttatattggctaggctctgcatcattagttctattgccgcaggattctcttcaagaagcaccagatcatcggcaaatcttaggtggtttaggcgagctccatttatatttagacctaagtggtcccagtttagctgtctaaagatgtgctcgagtacggcgttaaatagtgtaggggagagaggatcgccttggcgtacaccctttttgataggaaatgactctcctgttgattcaagtttgatactggccttgctagatttatatatactcttgagtatttctatatagttgctctggactccttgttgttcaagactccgccatatgtattggtgcctgaggctatcgaatgcctttgagtaatctatgaacgccaagtatatttgtttattgtattcattatacttctgtatcaactgttttattgtatgaatatgatcgattgtgctgaagccttttctgaatcctgcctgctctgcgggttggctttcgtttagcgttaatgatattctttccaggatcactttagcaaatactttatatatgtttgatattagagaaattggcctatagttcccaatctcgtctctagtgccttttttatgtagtaaaattatgtgacactcggtccattcttctgggatttgccttgtttttaagatttcattgaagagttttgccagcacaggactgatttcctctaaagttcccttaagcatttcgttaggtatcttgtccggccctggagctttctcgagtttttgggattttattgccttggttacctctgaggcaagaatctctggcactttgtctactttgttttcatttgcttcgtaaacttccggaattgtgtccaaccctgaacctaatttatcagaatacaataggcggtaatagtccgtggctagtttcagtattgagagtcggttaattaagttagattcgcgttggttcagtttcaatatccattctttcccgtgttcgcttagttgcttcagagccttttttgttcctcctgttctttttatattttcttctaaaacactatttcttttctctctacggtcttttcttatattctccttaattttcttgctaaggtttgcaattttttggcgtctttctttatcccctttatttgatattagtgttcttcttttatccaatagttctagtgtctccttgcttaaatagctttctttgctcttaggtttgctgactagcatctctttcaatttgttttgtaatcttctgtatttaatcccagtttctgcgtcggaagcaataatttctttgagtgattctgttttgggagaattaatggaatgactgtattctgacatttgacacagcttattattatttgctaggtttttcctggatcttttagattggtttaagcttaggcagcttctcaccatacggtggtctgtgttaaagttaaggtttttaactatgttggtatctgtgaacgcgcttgctttgttagttataataaagtctatttcagttttgaattttgcacctggggaggaccacgtccatttgcttttagtctttttgtgaaaacgtgagttcattaaaataaggttattttccatcatgaactctactagtttctgtccattttggcttcttttgccatgtccaaacttcccgattgtggattcttctccattttgtctttctccaacctgtgcgttaaagtcgcccatgattattatattgttattgtagtgcggttttattgcttctgagatactctcataaaaatattctatgtctgatatccctgcttgttctgttggggcatatatttggaagatggcccattttttcttgtattccggaaatttaaaatgagctaccgcaattctgtcagatactccttcaaaacccataatgtttttctttagcctagacttaataaggaagcctactcctttttgtccggtgtgttctcctttgtgaaacatgatatagtcgttcctctcttctattccttctcctggccttctcatctcgctgatgcctattatgtcccattttatgttggatatggctatttctaattcttttaggctctcttccgttcttagtgtccgagtattcagtgtacagacatgtatatttttatgttcggtgttctttttatcttgttttgtctgtattgttttctttttgtgcatattttttctggataggtttgcattaggttgtttcgagttgtaatattttgatttagatggagggtgtttgtcttcttcccccacggtgacaagccggcttggggtgcttgttatacatgttgtgtttggtaatgtggttgttgggattgtttggatccgggtgccgcatattagttttttgagggatttgataatgaagcggatctcccgcgctctatatagttcagtatatttgggcgcattgtttcctttgtcgcggttttagaagaggcgttgttcagggtggtcgatgtactttgggtattacttgcattgactttcttttgtgatggtgttttcggtgatcttgaagtctctcttttcctcttttctctgttgctttcatgtgttttctttattatcagtttgtcgtatttgatgaacgctatattccctttgttcttttcctcttcaacttgccgctgtagttgtttacgtttttctattatttctttagggtagtcctctttgatatttataccttcttgtagattacgtctatttcgcagaatcatgtgttttttccatgtagttgtgagtgagactacaacagggcgatttttatcgagctcttttttaccgatcctgtaagtattattaatttcgaaggtttcaaaatgtactcccatttccagtataatgtcttttatatagtctactagctccgtttctgttgttgcttttccttcctcgatgccaaaaaagacaaggttatttttgcgcttatccttatcagcgaatttcaattttaactctagctcactgattttatttttgagttgggtattttcttcggtgatcgctttaaacttttcgtctagtgctgccatgacattttgtgttactgtagttgttataagtttagtttgttggtctaacttttcatctaattttgccaatagaatttccatacttttctccatgtttggtattttttatattgctatggcagcacttgtttcgctgtgacaacactggttttgtaatggcaacactttttttgctgtggcagcacttgtttcgatgtttgttatgttggtactgtagtttccactctgtagtctatgtacaatttgcatcagatgacacttcacagtatttttgtagggtagaatgattacaatatgtggttgatttgttctcacaatttaacacttttctgtagttttttgctaatcgtgtaatttgttctttgttgcaaacaatagttcgtatgacactctttgttggcacttttttacactgtttatttttaattttccgcggagctctgtgacgtgcgtattgactgatcgacaaccggaaccggaatcctcaacataacattcctgatatttaagaatgaggcagtcgttcttagactcttcgtttccaggaagacgatacatcaatatgcggtttaataggctagagtgactatgttatcctagttggtaattaattcactagcataactatatgattatcgttaatctgattcgcctttttctttctttgtttgattgcttaacaatcgctctgggagcaatttggtttgactctattggtttagtcttttttttaatcatcatcaccccgatgtcaaaataatgttcttgttaacaatttcgactaacttaacgaggggtgaaaggcaatttggtataagcttaatagtatttatctttaattaaaggtccattttatttggtactagcgacccgccctcgcttcgctacggaaactgtaatttattattgatttctccactattcaatggatgttattatacatataaaccttcctcttcaatcgctctatctattaaaaaaaaccgcatcaaaatccgttgcgtagttttaaagatttaagcatacgtagggacagacagatatagagacagagaaagcgactttgttttatactatgtagtgatgattactatcaacagttcgatgtttttaattgaacttgaattaagtttaccccattcaaatagctgaagttttttttttttcatattttaaactttaaatggcttcccggtaaagttgcaaaaatgtcacttaaaatagacacccctcgatatattgtaaagcacaacatatatattaatgaattttttatttatttttcaggcgctcagcgtaaaagctttcggtcaccaccgtgagtattccttatgacacattattcccgcaaaaccattacagaaacttctcagaatgcatcctttttacataactctaacttaaacagtgctaatacacaaaagatgaacagtattttttttcacgcaggacttggcaattatatcgcaatgatttgaatatatatggttctatatatgggatattactatatatttatgtattacgatttgattttggatattttacgtgctgcctgaatttaattgtctatgcgaagaatatttcttggataggaaagttaaattctcgcgaattagtagaacgtccaaatttaaaaataatgtaaggttatttatatatctagagattaatgatccttatcgtccttaataatagaggtgaatatatcagataaaggaggagactgcgttatctcatcgaaagaccaacaaatttttgtcataaaacatttcttgaaaataaaatttgaaaagttatataataagatgtcgaatttccaaatcgtttacactaatcataatgcatttttttattgaacataacagcaaaatggatttaataagctttcgaactaaacaaaaggttacttctacaaaaaacaaaagtcttcatgtggaaatctaaatcgacataccgccgcttcggaatacagtctcgcaactcagaaaactcatatcaagggtttttttttatggtggacgagctcacagtccacctggtgttaagtggttactggagcccatagaaatcctcaacgtaaatgcgcccacccaccttgagatataagttctaaggtctcaagtatagttacaacggctgccccacccttctaaccgaaacgcattactgcttcacggcagaaataggcagggacccacccgcgcggactcacaagacgtcctaccaccagtaaaatggggtggaatgctatctggtttaagcgtcatttatctttggtgattaaaggtctattttagtagcagcaacagttcgatatttttaattgaacttccattacgtttaacgcactcaaatagcgcaagaagttttttttgtcattatattctttccaaatgtttaaactttaaatggctctcttgtatctttgcaaaaatgtcgcttaaacatttcgaccctcgatatgataaggctattgtacacacacattgtcttcataacattataatttaactatataaaaacattttgtataaaacaacaataccttacaaaatataacgttcgtggatacacttcatgtaggtgcgacaaacacaggcaattttaatttttttttattgcttaagtgtgtggacgagctaacagcccacctggttttcagtggttactggagcccatagacatgtacaacgcgccacccaccttgagaaatattttttttaaggtctcagtatagttacaacagttatattgcggatttgatttttattacacgttacgttattacttcaccgtggaagtcaatcgaaaacatttgttaagtacgtatttcattagaaaaattggtacccgcctgcgggattcgaacaccgttgcatcgctcgatacgaatgaaccggacggacgtcttatcctttaggccacgacgaataaaattagagaataaaaaaaattaaaatttaataaaaggattggtgaaatgtcttcggaaagagtttagttacgcgttaagaagtaatacaaagaaattcaatcatacgaaattcgttccagtttttatggcattagaacacctttttcaatgtcgtctgaatgttaatctatatatataaaaatgaattgctgttcgttagtctcgctaaaactcgagaacggctggaccgatttgcctaattttggtcttgaattatttgtggaagtccagagaaggttaaaaaggtagataaatatgaaaatgcgcggaattaaataaaaataacaattttgttttttctttaatgtgtcccccaaaagtttaggtcttttatttattgattgaggcactacggagtctgccgggtcagctagttgataatagaaacagtcctaagtaacgcggcattttatttatttattaggcacacaatacacattacaagctaaaaagatacataaaaaacacaagtaagaagaaacaaaatctgacttgcaacataagccatgtgcgcactaatccaactaattacgaattaagagggcttatcttttttttttgtatctaattcgaaataaaaatcggatttaagcatgtccgttaacttaatagacttcaacttaataataatctggatttggtcttgtcatggcaaagtttagtattttttttaatgcaaatggaaggttctggaacagaatgcgataatcggagaccacagggtcgttttgggcgcagtcgctgtttaagcaactagggatatatactaaaaaattatagccaaagggcatccggcttctgaaaacatccaaccaatttcggtactattttccatacaccatcaggccgtttgaggccaataagggctaatacatttatatacgcgcctgatctatttaagctttaccgcataaatacgtgaagtgttaacgcattgggagataacgacagaaggaagatcttccgccaggcggttgaaaatattctgtagacagaccatccgaatattattgttcggaacttgtatatacacaagcttatcttgaaaatgacgtgaagttcaggcatctatcagatatctgtcttttacgatagcttccgacacggtgtatcacacgagctcaccagaacccagaaacacttggaccgactaaactgtatacacattcagaaaaaacattcttcttttgtgaaaagaatattgaacttaacgaaaatgggcacaattatgaatttcctcacgcttacacaccacaaatgacgtacaaatcaaccatacactctcatgacaagaagcggcagcaatagagacggaaggcaattgaaaaagtaactaatgtcattctgtttgttgaacatttttagtaagtggcgacgtagctgaaaatagcaacgtcttcttccataaaccagttacgtaatagcagagacgaggatgtcccaatggcacatgatgttcacaattacatttcacattttttaataataatatttttaataaaattttaaattcataaattgactaagcgttttcacgtacccacacaagaggagtacactactggatccacgcacaagatacgccaagaacgccaaaacacaaagcaaaaccacaaactcgcctggaacatacaaacaaacagcagaccacactaacatggtaacacgcaaatgtgaaattaatccctctgtcttcactaatcttatattgattcgcagccggcaatcgagatacagtcgaagtcaaaaaccgaaagtacaatgcagctagcagtgaaagctcttacctcaacaaagataatgattcgataagtgccggagcgcaccgggccaagtccgtagagcagagtcaggataaaagcaaatatacatctggtccagaaggcgtgtcgtacagcggaaggtctcagaactataaagattccaagcaagcttatgccgattatcacagcgatccgaacggcggatctgcttctgccggacaatctcgcgacagcagcctgagagagagaaacgtacattacgtctctgacggtgaagcagtggccgcttccagtgacgctcgcgatgaaaaccgatccgcccaacagaatgctcaggccaattggaacgctgacggttcttacggagttagcgctgatcgaagtggttccgctagttctagacgccgccaagccaattactactccgataaagacatcactgctgcttctaaagacgattcacgtgcagattcttctaggagaagcaatgcctattacaacagagatagtgacggctcagaatccgctggattaagtgaccgtagagcttcttcctcgaaaaatgataatgtatttgtttaccgcactaaggattctattggaggacaagcgaaatcttcaagatcatctcattcacaagagagcgacgcttattataactccagtccggatggaagctacaacgctggtacgcgagatagttcaatttctaacaaaaagaaggcgagctctaccatctacgctgataaagatcaaatacgcgccgcgaatgatcgttattcttcgaaacagttaaaacagagcagcgctcaaatctcctccgggccagagggcacctctgtaagcagtaaggataggcagtactcgaacgacaaacgcagcaaatctgatgcgtacgtcggacgggacggcaccgttgcttactcaaacaaggacagcgaaaagacctcacgacaaagtaatacgaactatgccgaccaaaactccgttcgctctgactctgccgcttcggaccagaccagcaacagttacgacaagggctacagtgataaaaatacagttgcccatagctctggtagtaggggcagtcagaatcagaaatcgtcgagctaccgcgctgacaaggacggtttttcctccagtacgaatactgaaaaatccagatttagttcttcgaatagcgtcgtagaaacttcagatggaacttctgctagtcgcgaatcatcagcggaggataccaaatcatccaatagtaacgttcagagcgatggtgagtattaaatgcgacacaaaacacataatcaattataaattatcatggtctcatttatcatcgttttaatattattaagtaactcgtcgccatctgttcaaacagttataatgaacgcgatgtttttccgatcattggcttttaacaaaagacatcggtttgtttcattcgttattagaggttaatcgtgtttactcttttaatttattgataacacaagtcattattgtgtaagaagtcgataaatctacacatacatagattaatgggaataatgataaattacccgcatttaaattaaaattaaaacttgtatttcgaatcagcagtaactacctacttttgggattttaaacaaatcttgcttctcttctgggtgcttaagatacaccaagaatatgaagccttttcttcatggcatgcactttcttcattacgctatttaattgtctgctttttccgccacagtattagggtcatacacaaatagagaaaaaaaaagtgcagcaaaaaaaatatctctaaatcagtgtgaaacaaactatttattatttaactattgattatcaatcttttaatcactgtaaccgagatcaatgtgtcttatttttggcataatcgcaatttttatatttttgcggaccacagaagagacaatttagtctacaagagatgaaaagctgtgatgcagcgtaagctaaaatatattaagacaacataatcaaacacatgtagccactgatataaacgaataccaaaacgcaaataaaatgtcaaaataatcacattaattggaatataaaaaataaaataataatgtctacagaaaggaaaatcccttcggtggacttatgaaacaaaacaaaatttcgaatgataataattttacagccatggtatattattcgtattgaaccccctgaacgaaatcaataaaaataaaaataaatgttttcgtttcctctatttacattgattagatttataattttttctattgcgaatataaaatgtactaaaaagtttgaggctctaacttagaatatcaaatgttgccaaaatcaaacctctttctaaccaactttaaaaaaggagtaggccctcggttcaactatttttctacttactactttctctaattagacctactaaggtatgtttcttacacattttctgtggacatagtcttttcggtggacatcgggaccacaggaaagatgtccacagaaaagatgtattataaaaactcttgcgtaaagaaattaactttaaggttagaaatcaagcaacatgcttttcatttacttgtactaaatgctcatataaaaacaataaatatacatttttatagataatatttacatgtctataattggaacaggaaagaacacttgatcttttacgtttatgtgtgttttggaataaaaaaaatattttcactcgcactgggatcgaaacaacaaaatatcaaacgccggaatggccactatttagtgatgccaacagttaaaaaaataataataatgcggtcgaataaattaaaatttgatgtcgttttatcgtgtccacagaaaagactcaatttgatgtaaccataggtttattggatttattgtatacagatagtaacgatatattttttttattctcaatattatcatctttattatattgtttgtattacaagtaaataattttaatatatagggtactttaactatttttaaacatggctcaaacattataccttcgtcttgtaacagtccacaggaagagattttttgatgtttaacgccatcaaaatgaacttatgaaatgtaatattttacaagatacgattaaaatgagtagtaatatagtacctttacgatttaaatactttcaaatattcgccaatttgctcttttagatgatacattgctataacactttttttaccctttttgtgtatgacccattagtcagctaacctttgcctgccgtgtaggtcaccggtgccctacgcggcgcactgaagcaattatttcgtattcagtttctaaggcgccggaatatgtagtagactctggaaaagacgaatccgaacgatactgagaatgaatcgtatttctaagagacctaaaagacaaaaacgtacattgaaaaacaagagaagataaagttaggctatccaggtgcttagtaacaggaatgggaataattactgtagtgcgtcacgtagggcaccggtgacctacatggcatcacacatatatgttaaaaatgttatttctccattattacatttgttctttaacaatcaatgaccgaaatcggcagtgctacatgcatatttcaactaatacaacattttgacattagaccaaactaataatattaatgtatgaacgattatgatcgggtctcgatatgtgtgaaaattattttgattactaatcggattttgattactacttgtttttgctgaaaattacattgaaacatctcatctgatcaaatgaagttcgctatgtatatattttatttggttcgttcacacaaaaatatacaacggttgaaagtaatataaaaacgcaataagtaagactctaacttaaataactggaacacgaaataaagacgagactgagacacggtcggtcagtgtgggtgggccttatcatagcgatcaaagtcacccaaagaactatggaggattatgctcagagtttccttgctagatcggatcaggaatgaggagaaacgtaggagaaccatgataactgacatggccaaaaggattgcgaggcttaagcggtgttttttttttttctacataatctgatagccttgagaggctatatcagcgtcgccttaaccagtagatgagctcacggggctcaaacctgatgacgttgctaacacgaaccctagcaagagccgtgcttcgcagaatctaccgccggatcggaaacgcgacccactgagaagatccggcgagaaactcagtgggctaagtggtagtggacagggcgcattgctagtagaaaagatggccgcaatggccaaaaagctctagagttgcgtccgcgtacccagtagcggatttgcagtctcggctgccctaggccccaagccaccaaccgcccctttctcagcatccaccatatagactacataatgaaattgctttattgccaacttttatttaaatagatttagaaatttaggaaaaaaaattagttttatttcgcaagaatatggtacatgagttcttacaactagttatttgctctaacatactctgccaacttggcatacttggttatgataacttttacacgagtgttatcaactttcaacacttaattgcgagttaaatatttgaatatctaattaaacacaattaatttgtacggagctacttgttatcgcaagtgtgggtgcgaagtgtgggtaaaagtatgttttttgacacgattggggccgtccctaaatctggccttagggtaaatcttagggtaaatccgccactgcgcgtaccggaagatgaatcgtggatacgcaaagcaaacctacgatctgttgaaggttgcgggatttcgctgggtacagacagcataggaccggtcattacgacgaggcttggagtagacctatgtccgtcagtgagactggtagatagataaagaccagcctttattcattcttgttcgtttaaggcctttaatggaccaaatcaatacaacggttttttcatgcttagcttgttaggttataatattattttttatttatattaggaatcgctccttagtacctatttatgggaatagcttatcttaccttcttgatgccttcctaagcaagaccattttatctaacaagataagctgttaccacagtagttcatttctattaatttgtttcataaggggttatcattataaatacgattgctatcggcatgtctccacggtctaacagctaaaacgcccacaggaagagcctttatcgtaattcgatccgcgtgtagatacaaatctcaaagcgattgtgtatccagcgtgtattcataaatgaaatgatgacgtcataattttttatttatttcgtgtgcactcatacttcgtgtgcacaataccaaaaacgccgtcacttttcatgtatttgtcgtgtttgtttgttcgaaaaccataagaagactaagctttttattgttacagattttaactttcagttacttttctatgtaatcgaatattatcagtgcttgggctccgaagacggtatctttctaacaagagcttattttaagacgccgctttgaggctacttaaattactaaataacgtaataagcacgagaatattcggatccggtagcagatgcattcgtgaagcagttgtccttttttttgagctgtaattttttttttattgcttagatgggtgggcgagctcacagcccaagtgttaagttactggagaccatagacatctacaacgtaaatgcgccacccaccttgagatctaagttctaaggtctcagtacagttactacggctgcctcacccttccagccaaaacgcattctgcttcacggcagaaataagcggggtggtggtacctacttgtgcggactcacaagaggtcctaccaccaagactctttcggaggcgatcgggaaactgttagctaatcccaactctcatggctgagcctttgctggcccacctatcctggtgaaactggaaaggcctccaggccaccagtaatctttcaatcataaaaaatcacaaacagtgtaaataaaataaaagcgtcctttgaagtaaccaaaatttggaatacaaacatttcttcatgacttacattttgaatgattaataagaaatgattagtttttaataattaacttcatgcttcctcacctctcaggttaaccataaagtattattatatgcgatttttaacttcacgttaacaggggaacgagtgcgcaaagaaacatttcgtatcaggtattgggttcagagcttcattctcattagtacagtcatcgctttgacattttggcttaacatccttatcagcgtgtcgatcttcattcgccattcgttccataccgacactcattaacaggacttcgtctgagtgtttcatgctctaattaatcgtatcgccaaaatcgtatctattcacatccattaacataccgaaaacgaaccataaaccataatggtgtcgcggaaaaaccgatgtaacgttttataaatatcggttttccagaaacaggcgaagaagaggaattgttcgatgttgtatcttaccagaaaattgaagatggcaagcctgtaatcataatgaaagttataccagtcggtaagataaacgttgatgaagatacaataatgtcgtttaattaaagacaaatgaaactgcattgcgcattcggtacagaaagtacgacaggcatgaagtaatcaatagttaaaggtattgaataggtacagtagatatgatgtctacttaacgaaatacaagatcgacaatgctattaagatattaagttaaaatgtcaaaaatctcaattattatctaagtcttgagggtaggaaaaatccgataaatgactatatgtcgggtattaggtattactatcgaaatttcggaagtaatttatttagcgtacgttttatagagaaatccgcgtcccaatcaagttcttcgcggtcatctcaggagtctgcaagctatagcagcagcagcagttcatcgagtaagttcattagcgttttatgaagcgaacacgaattaatcggccacaggcctgacatacgcctttaaatcctacgacaaatgaaaaagattctcttaaagaaggcaaagaatgacaattcttttctcaaagcacatcgttggtattgcgttgtgaactcttgggtgtccggcatctaattaacctaagaaggcgcatctacagcgcttcctcggttctacagcaggtgtaaattatgttttagttagacattcctcaagagtttttttgatttgttttttattaatatttgaatagcttccttctgtatcgtgataaacaggaatattggggaatagctttagtcatgatcaaatcggtattgcgctttaggtccacgtccactctccatatggaccggtccaggccgtgatatacccttcttcagaagaaaatactcaggcgctcgctctgttcctaagccaataccaataccaattcaatcaaaaggttcaacggaacgaccacagcctacagggcaccgttcaggtacttgtgatattacgtcaccacatgtcatcagcaatcatacgatcacaagtcatatcatttataatctgtccacattcatagaccagagaactgacgataaacttaaagctattcctttttgaaatttaaagcagaagaatcctcatcctcgagctctagggctgcttcatcaaccgacgcttctagcaacactgattcaaactcaaacagcgcgggatccagtacatccggcggtagcagcacttatggatacagttccaacagtcgtgatggaagtgtatcgaccaccggcagttccagtaacactgattcgaattcaaacagcgtaggatccaggaaatccggcggtagcagctctcatgaagacagttccaagagtcgtgatgaaaatgtatcgaccaccggcagttccagtaacaccgattcaaactcaaacagcgcaggatccagtacatctggcggtagcagcacttatggatacagttccaacagtcgtgatggaagtgtatcatccaccggcagttccagtaacaccgattcaaactcaaacagcgcaggatccagtacttctggcggtagcagcacttatggatacagctccaacagtcgtgatggaagtgtatcatccaccggcagttccagtaacactgattcaaactcaaacagcgtaggatccaggaaatccggcggtagcagctctcatgaagacagttccaagagtcgtgatgaaactgtatcgaccaccgacagttccagtaacactgattcaaattcaaacagcgtagaaaccaggaaatccggcggtagcagctctcatgaagacagttccaagagtcgtgatgaaaatgtatcgaccaccggcagttccagtaacactgattcaaactcaaacagcgcaggatccagtacatctggcggtagcagcacttatggatacagctccaacagtcgtgatggaagtgtatcatccaccggcagttctagtaacactgattcaaactcaaacagcgccggatccagtacatcgggcggtagcagcacttatggatacagttccaacagtcatgatggaagtgtatcatctaccggcagttccagtaacactgattcgaattcaaacagcgtaggatccaggaaatccggcggtagcagctctcatgaagacagttccaagagtcgtgatgaaaatgtatcgaccaccggcagttccagtaacactgattcaaactcaaacagcgcaggatccagtacatctggcggtagcagcacttatggatacagctccaacagtcgtgatggaagtgtatcatccaccggcagttccagttacactgattcaaactcaaacagcgccggatccagtacatcgggcggtagcagcacttatggatacagttccaacagtcatgatggaagtgtatcatctaccggcagttccagtaacactgattcgaattcaaacagcgtaggatccaggaaatccggcggtagcagctctcatgaagacagttccaagagtcgtgatgaaaatgtatcgaccaccggcagttccagtaacaccgattcaaactcaaacagcgcaggatccagtacatctggcggtagcagcacttatggatacagctccaacagtcgtgatggaagtgtatcatccaccggcagttccagtaacactgattcaaactcaaacagcgccggatccagtacatcgggcggtagcagcacttatggatacagttccaacagtcatgatggaagtgtatcatctaccggcagttccagtaacactgattcgaattcaaacagcgtagaatccaagaaatccggcggtagcagctctcatgaagacagttccaagagtcgtgatggaagtgtatcatccaccggcagttccagtaacactgattcaaactcaaacagtgcaggatccagtacatctggcggtagcagcacttatggatacagctccaacagtcgtgatggaagtgtatcatccaccggcagttccagtaacactgattcaaactcaaacagcgccggatccagtacatcgggcggtagcagcacttatggatacagttccaacagtcatgatggaagtgtatcatctaccggcagttccagtaacactgattcgatttcaaacagcgtaggatccaggaaatctggcggtagcagctctcatgaagacagttccaagagtcgtgatgaaaatgtatcgaccaccggcagttccagtaacaccgattcaaactcaaacagcgcaggatccagtacatctggcggtagcagcacttatggatacagctccaacagtcgtgatggaagtgtatcatccaccggcagttccagttacactgattcaaactcaaacagcgccggatccagtacatcgggcggtagcagcacttatggatacagttccaacagtcatgatggaagtgtatcatctaccggcagttctagtaacactgattcgaattcaaacagcgtaggatccaggaaatccggcggtagcagctctcatgaagacagttccaagagtcgtgatgaaaatgtatcgaccaccggcagttccagtaacaccgattcaaactcaaacagcgcaggatccagtacatctggcggtagcagcacttatggatacagctccaacagtcgtgatggaagtgtatcatccaccggcagttccagttacactgattcaaactcaaacagcgccggatccagtacatcgggcggtagcagcacttatggatacagttccaacagtcgtgatggaagtgtatcatccaccggcagttccagtaacactgattcgaattcaaacagcgtaggatccaggaaatccggcggtagcagctctcatgaagacagttccaagagtcgtgatgaaaatgtatcgaccaccggcagttccagtaacactgattcaaactcaaacagcgcaggatccagtacatctggcggtagcagcacttatggatacagttccaatagtcgtgatggaagtgtatcatccaccggcagttccagtaacactgattcaaactcaaacagcgcgggatccagtacatccggtggtagcagcacttatggatacagttccaacagtcgtgatggaagtgtatcatccaccggcagttccagttacactgattcaaactcaaacagcgcaggatccagtacatctggtggtagcagcacttatggatacagttccaacagtcgtgatgaaagtgtatcatccaccggcagttccagtaacactgattcgaattcaaacagcgtaggatccaggaaatccggcggtagcagctctcatgaagacagttccaagagtcgtgatgaaaatgtatcgaccaccggcagttccagtaacaccgattcaaactcaaacagcgcaggatccagtacatctggcggtagcagcacttatggatacagctccaacagtcgtgatggaagcgtatcatccaccggcagttccagtaacactgattcaaactcaaacagcgccggatccagtacatcgggcggtagcagcacttatggatacagttccaacagtcatgatggaagtgtatcatctaccggcagttccagtaacactgattcgaattcaaacagcgtaggttccaggaaatccggcggtagcagctctcatgaagacagttccaagagtcgtgatggaagtgtatcatccactggcagttccagtaacactgattcaaactcaaacagcgcaggatccagtacatctggcggtagcagcacttatggatacagctccaacagtcgtgatggaagtgtatcatccaccggcagttccagttacactgattcaaactcaaacagcgcaggatccagtacatcgggcggtagcagcacttatggatacagttccaacagtcatgatggaagtgtatcatctaccggcagttccagtaacactgattcgaattcaaacagcgtaggatctaggaaatccgacggtagcagctctcatgaagacagttccaagagtcgtgatgaaaatgtatcgaccaccgacagttccagtaacaccgattcaaactcaaacagtgcaggatccagtacatccggtggtagcagcacttatggatacagctccaacagccgtgatggaagtgtatcatccaccggcagttccagtaacactgattcaaactcaaacagcgccggatccagtacatcgggcggtagcagcacttatggatacagttccaacagtcgtgatggaagtgtatcatccaccggcagttccagtaacactgattcaaactcaaacagcgcgggatccagtacatccggtggtagcagcaattatggatacagttccaacagtcgtgatggaggtgtatcatccaccggcagttccagttacactgattcaaactcaaacagcgccggatccagtacatcgggcggtagcagcacttatggatacagttccaacagtcatgatggaagtgtatcatctaccggcagttccagtaacactgattcgaattcaaacagcgtaggttccaggaaatccggcggtagcagctctcatgaagacagttccaagagtcgtgatgaaaatgtatcgaccaccggcagttccagtaacactgattcaaactcaaacagcgcaggatccagtacatctggcggtagcagcacttatggatacagttccaatagtcgtgatggaagtgtatcatccaccggcagttccagtaacactgattcaaactcaaacagcgcgggatccagtacatccggtggtagcagcacttatggatacagttccaacagtcgtgatggaagtgtatcatccaccggcagttccagttacactgattcaaactcaaacagcgcaggatccagtacatctggtggtagcagcacttatggatacagttccaacagtcgtgatgaaagtgtatcatccaccggcagttccagtaacactgattcgaattcaaacagcgtaggatccaggaaatccggcggtagcagctctcatgaagacagttccaagagtcgtgatgaaaatgtatcgaccaccggcagttccagtaacactgattcaaactcaaacagcgcaggatccagaacatctggcggtagcagcacttatggatatagttacaacagtcgtgatggaagtgtatcatccaccggcagttccagtaacactgattcaaactcaaacagcgcaggatccagtacatctggtggtagcagcacttatggatacagttccaacagtcgtgatgaaagtgtatcatccaccggcagttccagtaacactgattcgaattcaaacagcgtaggatccaggaaatccgacggtagcagctctcatgaagacagttccaagagtcgtgatgaaagtgtatcgaccaccggcagttccagtaacactgattcaaactcaagcagcgcaggatccagtacatccggtggtagcagcacttatggatacagctccaacagtggtgatggaagtgtatcatccaccggcagttccagtaacactgagtcaaactcaaacagcgaaggatccagaacatctggcggtagcagcacttatggatacagttccaacagtcgtgatggaagtgtatcatccaccggcagttccagtaacactgattcaaactcaaacagcgcaggatccagtacatctggtggtagcagcacttatggatacagttccaacagtcgtgatgaaagtgtatcatccaccggcagttccagtaacactgattcgaattcaaacagcgtaggatccaggaaatccgacggtagcagctctcatgaagacagttccaagagtcgtgatgaaaatgtatcgaccaccggcagttccagtaacactgattcaaactcaagcagcgcaggatccagtacatccggtggtagcagcacttatggatacagctccaacagtggtgatggaagtgtatcatccaccggcagttccagtaacactgattcaaactcaaacagcgcaggatccagtacatctggcggtagcagcacttatggatacagctccaacagtcgtgatggaagtgtatcatccaccggcagttccagtaacactgattcaaactcaaacagcgcaggatccagtacatctggcggtagcagcacttatggatacagctccaacagtcgtgatggaagtgtatcatccaccggcagttccagtaacactgattcaaactcaaacagcgcaggatccagtacatctggcagtagcaacactcatggtgccagccatggtttggcatcatctggcggtagtatagcatcagcaactagtactcctgacatacttaccataggtacctgtattgtatttgaaaaaactttagctttttttttatcctacctgagttggtagctttagaggattatgttagcgtcaccgagcgaataggtgagctaacgggacttctaatctgtggaagtttagctaattatacaaatttgtaaacagtgacaaattaccaactgggatcgacgtaggaaataatgttatctatcatattttatgtaatttaaatactgtctaagctctgtattcattttaccatttttaaagttatacatgaatccatatgactcatttttgtatcggcaattagaaaatgcaaaatcatgcaacatttttaccgaagcggccggatattccttcgattaactgagaagttccgctaaagtcttgagagagaccctatctgattgaatgtctctcagctgatcagattaccgtacgataccgaactccttatcaaaattaaaacattgcagcttatattgtgcaagtaatttcgcggataccgaaaaccaatccgtaattttgagactgttttggacagctttattttcacttatgaagtcgacatggcctacgggataagacacggtgcattcgttactagcggtgtcgatgttcgaatcccgcaggcatgtgccaatttatcgaatgaaataagcaattaacatattttcacgaattactttcacggtgaaaataacatcgtggaatatagacaataacaatcccgtactagataattgtaatcgaaaaatcaatagctttaataaaccctgcctacttataccgtgtggcaataatacgtttcggcagccgttgtactgaaaaactgagacttaaaactcatgtgtcaagggtgggtggcggaatttacgtcgttgatatctatgagcgccggtgaccgctaaacaccaagtgggccgtgagttcgtcagttcatccattatagcaatcattaaaaattacgtttcatttctttccagcactaagtgaagactcttccgaggtggatattgatcttggcaatttaggctggtggtggaattcagacaataaggcacaaagagcggcaggcggcgcaacaaagtctgaagcttcatcatccactcaaggtagattttgtgattgtcgaataacaatcttaatgtgtaagggattatcaagaaaatacccaatattacttcaaactttggtatttttgggaaagacacgaccgcattttttgaaacttatgctgtgtaaggattgcatactttcaatccgacaattacgttacagtaaaaaggtgaagtttaattttattgagtctctgaaacaaccccataagggggttgcctccatcatcatcattagcccaccgacgtccactgctatacaagtatccccaaagtttcgccgcaaagaccggtcctacgcagcccggcatctagcgtattcccacaacctttagcagttgtcgttcaaactcattggaggtctacccaatctacgtttttcagagcacgatcgccaatcgagaactcgccctcgtactcccgcccaaattgaatacgacagaaaaagccttttttactatgaattggttccgatagacccgagtagatatgacaaataatcaccacccgaaatgatgaccagtgaccacgatcacccggccaagaatgcaagcgactgagaagaagtattgtacacaacccaccgagtttatcggcgattcttctcagggggtcgcgattccgatccggtggtagattctgcgaagtactgctcttgttagggctagttaaaaaaaatctcaggttgagcccgtgagacctgagggggtgaggccgcgcggcgcgctaccagcactctagcgcgctgccgtggaatgaatagagcgaccggtcgacggtgtatcgcgtcccgaccgggcaggctggttctggtccagcggggtattccgggacaccagcggcaccgtctgggcggcccgacgggctgccgtaccgagacggccgacgtttcagagccttcgattcgcctcgaaggctccgtcggctgggcgtccttggggtgagccgcgccgtctggttgtagtgttgaccgcggtagcccccctacctcatccgggttctgacctcggaggggatcggacgtcgggtgtaagagtgcaggggagtcgtttagtgggtgggtcctaaaatccttgggcccgcggtctgctcacaacaccatgcagatcgttgagtctcacataccccgcgcgccccttttgcgcggggacctcgtaggagattcggccctctacccgaaaaaaaaaaaaaaaaaaggttgagcccgtgagctcacctacccgtccgggcatagctggaatagcccttcagtttttaacatttctgacaaacgggcaggaactgcctcctagataccttggaacaaatagccccttagactaccagcgaataggtaggaggaaaaaaagtactgtatgtacaataatcaaaaactatcagttcaatatattcatagacctcttatgttcaaaagagtgcaaaactaaaattactttgtcgttttattaatttcgtttggtttattttaagtattcgacccttaacatttctcgagaataccatcgactaaggttgtttgttaaattcgtcgcgtcgatcgcgtaaagctaactcaaatttgctcaattatgcagctaactaaaaaaaattgtgtgaacttgaaacagcgcccctggcggcaaacgaagggaagctatttcaactccatacgaattggagttaacttttacgcgatcgagaacgttaaaaaaactcgccctaagcacacagtcctgtttaatttatttattgcccgtgtccacatattaatagtgctttgtgtacacagtcggtggagaatggtaacagtagcatggaatatttagtatagagtagtgttctatggtatgacgcataagtaacttagcataaacataacttctaactacaaataattgtaacacacccaatgtttcgactaattacacaaaaaaaaattacagctactacagtcagtggcgcagacgacagtgctgattcttacacctggtggtggaatcctagacgatcaagcagctcctcttcatcagcaagttctagcagctctggctccaatgttggtggttcctctcaatccagcggtagcagcacttctggaagtaatgcccgcggtcatctaggaaccgtttcgtccactggcagtaccagtaacaccgattcaagctcaaaaagtgcaggatcccgtacatccggcggtagcagcacttatggatatagctccagccatcgtggtggaagcgtatcatccaccggcagttccagcaacactgattcaagcacaaagaatgcaggatccagtacatccggcggtagcagcacttatggatatagctccagccatcgtggtggaagcgtatcatccaccggcagttccagcaacactgattcaagcacaaagaatgcaggatccagtacatctggcggtagcagcacttatggatatagctctagccatcgtggtggaagtgtatcatccaccggcagttccagcaacactgattcaagcacaaagagtgcaggatccagtacatccggcggtagcagcacttacggatatagctccaggcatcgtggtggacgcgtatcatccaccggcagttccagcacaactgatgcaagctcaaacagcgtaggatctagtacatccggcggtagcagcacttatggatacagttccaacagtcgtgatggaagtgtatcatccaccggcagttccagtaacactgattcaaactcaaacagcgcgggatccagtacatccggtggtagcagcacttatggatacagttccaacagtcgtgatggaagtgtatcatccaccggcagttccagtaacactgattcaaactcaaacagcgcgggatccagtacatccggtggtagcagcacttatggatacagttccaacagtcgtgatggaagtgtatcatccaccggcagttccagtaacactgatgcaagcacagaccttacaggatccagtacatccggcggtagcagcacttatggatacagttccgacagtcgtgatggaagtgtatcatccaccggcagttccagtaacactgatgcaagcacagacctggcaggatccagtacatctggcggtagcagcacttatggatacagttccgactgtggtgatggaagtgtatcatccaccggcagttccagtaacactgatgcaagcacagaccttgcaggatccagtacatccggcggtagcagcacttatggatacagttccgacagtcgtgatggaagtgtatcatccaccggcagttccagtaacactgatgcaagcacagaccttgcaggatccagtacatcgggcggtagcagcacttatggatacagttccaacagtcgtgatggaagtgtatcatccaccggcagttccagtaacactgatgcaagcacagaccttacaggatccagtacatccggcggtagcagcacttatggatatagctcaagcaatcgtgatggaagtgtattggccactggcagttccagtaacactgatgcaagcaccacagaagaatccaccacgtccgctggtagcagcactgaaggatatagttccagtagccatgatggaagcgtaacatccaccgacggttccagcacaagtggaggagcttcttccagctcaggtactctaaaaagtcacgttacttcctctttttaatcactgtcaactcaaactggatctatctatttttccatttcaataaactttaattttatttgatttaaatttgggcactaccgaacttcgcgcgatcactctctaggtaaatccgaagatatccttaaaattccaccgaaataggtccagccgtttcaaagtatatagggaaaataggtttacgcattggttgctcctcattagtgatctggaattgtgaccggaattgttgtcagtggaagtgttggcaggtctactgtcatagccaatctctgcttaatactgtaaatgtgacagtgtgtttatttatttgtccttttctttcacgtcgagacgaaacaacatgattttttgtaaagtaattaatagttttacgtcaggaaagtgacataggctaccttttatccagtaaaccatctcattacccgggaaaatactctgacattttacattgatcacgcggacgaagcagcgattaacagctagttcgtttattaccaaaacacttttgttctgttaactagtagcaggcctaaaactcaattggtgggtacagaaagaaattttctaacactagccccagcaagttaatttataaggatataacctgttttcaaaatatggtactgttatatcatatactccaagaggaagctattatttttaatgtagagaactgagccattaatgagatactgggccattgtttacatgtcccttgaatgaattcatattccaataagcagcaattgaaatcacgctgaattagaatagtcaaaatgaatacgtctttgaaaacgtataatgtgttggttttatatattacagcgtcaaccgccaaaagcgacgccgcgtcatctgaagacggtttctggtggtggaatagaaggaaatcaggatccggtcacaaaagcgctaccgtacagtcatccacaaccgataagacgagcaccgacagtgccagcagcaccgattccacctcaagcacgtccggggcaagcacaaccacttcaggcagttcttctacctcgggcggttcaagtacatcggacgcttcctccacttcgtctagtgtttccagaagtcatcgttcaggcgtgaacagacttttacacaagcctggtcaaggaaaaatatgcctttgcttcgaaaacatattcgatattccttaccatctccgtaagaatatcggtgtttaatacaactaaacacgacttggagtattccttgtagtgtttaagattttaaatcttacttaatgacttcgaacgattttaacgataactttctctttgtttaactttaatcagcatacataaaaagccccggttttgtatcgggaagaaaaaaaatgtaattgtgttgcctagataataaacgtattatcaaag

SEQ ID NO: 2 - DNA, 12735 bp, Bombyx mori
cacgagtcatcatgaagatcccatacgtcttgctgttccttgtggtgagttgctttcgtttttgatatgctggttcctcaggagtctgtactaatgcttctgtttttattgtataaatgtgagcacttcacggcctacgtaaccagctggttacaatcaccgtccacgccgaaaaaatgaggcctgtatctaaattgtaacataatttttgcacatttgattctcatcccacgattttatttatctttcattcatttttactggtggtaggacgtcttgtgagtccgcacgtgcaccacctcacctatttcagccgtgaagcagtaatgcgcttcggtttgaagggtggggcagccgttgtactttataaacggagaccttagaactcatgtcccgagatgggtggcagcatttacgttgcagatgtctatgggctccggtaaccacttaacattttttttttttcttttttttttttttttatcacgctacgttaattggtcccgtgataagttcgtaaagaacttgtgttacaggtaccagataacggatataaatgtaagatttttattatacatatacatatatttaatagacatccataaccctggaaaagacatttatatttatcatacaaatatcttcccttggcgggattcgaacccgcgacccccttgtatagtgaccatgtcacttaccactacaccagacggccgtttaacattaggtgtgccgtgagttcatccacccatctaagcaataaagaaaaacccaaaatttataatttggcgtaattactggtggtaggacgtgttttgagcccgcatgggcaggtaccaccactctgcctatttagtagcagtaatgcatttcggtttggtggcagtcgttgtactgtaaaaccttagagctcatttcacaagatgggcggctttttcgttgttgatgtttttgagctcatgtgatggagctcataacaccaaatgagccgtgaactggtccaccctacttatcaaagaaaaaaggcgatattccttaagtggttaccggagcccatagacatctacaacgtaaattccgccacccaccttgagatatgagttccaaggtctcagtaaaattacaacggctgtcccactattcgccaatctcctttttaattatgtcacaagaagtcaactgccatactaataaatgaaaaattattatttttcattaaaaaatgatttactctaattagctagtttgttctacgaaattccaatcaaggaaattgtgttgaaatgctcggagacctttataatatcctcactcatgacaagaccctttctaaatagaccattctcttcaaccccttcaatccctgtccaccaaatgctgagtgtaggtctcttcaaatgtaccccattctcttcggtctcttgcttttcgcattcaattccttccaaccactttgaccaggtcatcggccctcctcgttggctgtctgcccaggctacgacatctccacatcagtatttttgtgccccactgattatcaggggcgttaacatggccgtcaaataagccattttagcatatggaccctggattcgagcagtgtatgatttatttatatttcgtttccagggcgtggctgtggtcaacgcattgcccagtaagtaattatatcgtgttaaagttactttattattataataatactgtcttgtttaaatcgtccgcgcggagtcggctagtagcttggaagtgcacagttgaatgataataattttttaactagtttaaaataaagtgaaactcaaagattcgatgcccaataaatttaatactgttaaaacatttgcagatccacttttcgggggcttagttaaatcgcttagcaaaaaaaaacaaatttttgaggacaaatttgaaaatctcaaagagaatgtgggtgaaaaatttgaaaatctcaaagaaaacgtgggtgaaaaagttgaaaatctcaaagagaatgttggtgaaaaattagaaaatatcaaagagaaggctggagaaaaatttgaaaatctcaaagacaatgttggagaaaaatttgaaaatctcaaagacaatgttggagacaaattagaagcagctaaagaaaaagctggagaaatcaaaaagaaactagtggatgtcggcgaagatctgaaagacgagctcacggaagacaaaaaaataaagatatccatctcgaaagacgaaggattgactttagaaaaagaaggatacaagtctgattacgatcgtaatgaatatgaagaacgcggatctgaacaccagggtgggtagtacttctttagtgactaaacaataaactaattcgaccgtgggagataattattagaataagattttagcggaaacgcaaggtgcgaagttgcgtgaatcgtttgtggttgaaaacggtggctcgtattatcgcatcccccggatcccctttcggtagcaaactaacccgtatccactgctcactgtgttcctcgcgggatcttctcagtggattgcaacttcgatccggtagtagaagcactggtcttgctaaagctagtgttggcaaattctctcaagttgagtccgtgagctcacctacccgtccaggcttggtcggaataaccccttagactaccagcgaataggtagggaaatgcaaaaaatggtaataattataattacattttaatcttgcctttattatttacatttaatatttattacccgtttcgaataccatgtatgtagagactttaatctaatttagtacagtatttttttatacgaacccgcacgattttttgcccgatattcaaatgtagacaactaagatcttagtcgcttgctaattaaaaataattctattatttatgtacgagatataagactatgctatacttagtgtactatagtgataactgctcagcatcggcagaaccgtctgttgtatttcggaatctaaatctagaaaaggaatcagacatttggccatcatcccgggccggtggttctgactcaaccacccaggacttaactaatttatttttctctacagaggataacgacagcgacggctcatacagcaaaggttccgaatacgaaaaatacggtgaagaggaaaagtatgaagaacgtaggacccacgacaagttctccattggcaagaacggtattagtgccgagcgaacgaaatcaaagagaggtgaaagaaaagaagtcgaaggcgaatatgagaaagactacgaaaggaaaggtgcgaattgttgtttgatcgacgataaattacgattttttttccgagaataaaatcggttgacgatagtcagtggaaaattttccagtcacagagtgtcgaatgaaaaagaaaatccaaactaatgtacatcaaatttctgaatccttcgaatgactagtagtcaggtcgatattgtgagtctctgcccgcggtcatcagtcagtcctacctaattttgccaggaagagctgccttccaacttaagccacgcgtcttgaggaaaccttccggacgctgctggaaacctttcaactccccgctcggtgtacgaactaaaataatcaacttacatatcacttagacgccatgacttcaatgaatctttttcatatgttcatttattcttatataaaaaaatggtaatgtgtatacatatatggtaataatatatgtgagcaatttttaccgacttcaaaaaaaagatggttctcaactcgactgtttttttatgtgtgtgagtcagaatttttttctacttgaagcgatttcgatgattattttatgatgatgacagtgagataattatccttaacaatgcacgattcaatgtttcattgttcacgttaattttattctaatttcattttgttgggagtcctatcttgttttttgttatcctttaatttttgaattagaatttttaatattacaatttgcggatccttatacttagtactgttcccgatcgtacgacacgagctgtttcataaagcagccttcctaggcccaacattcagataagagtcgaagtaatcatcataaatccatttctagagaacaacggcggatcctccgaatattcagagagagaaagagagagtttggaaaaatcaaaagaaaggtacggcgagcagtcgtctaagtccttctcgttgggtaaatccggcttgaagaagcaggataattcaaaatcgtattccgacaaggaagagtcgaaactggaaaaggaaaagaaatatggtgagcttataatacccccgaatcgaacttttgtggagacaattcaaaaccgtttttaagcgtttggacaggtcgacaggaatggttcgcgaagttttattttaaaactgaccatgactcgaacaggtgataggcagttcgtgttttgtcatatggcaatgtcaaatggcagtattcatttcaaaaccaaaagttgtctcgtagtcaattgatcgcattgcacgacccaatatgtgtgagaacccttgggctaattggtactcattttgagtcaaaagttttctattttattcttttattaatcttgtagtcaggcgcgtgtaaaccacatgatggttgttttattgcttagatgggtggacgagttcatagcccatccggtgttaagtgcggtggtacctaaacggagagtactcagcggtaggcagtggcttgactctacccctaccattgctaacctccatgatccggtaaccactaattggtggttggacctctagtgagttcgcacgggtaggtccaccctgcctatttctgccgtgaagcagtaatgcgtttcggtttgaaaggtggggttgccgttgtaactatactgagaccttagaacttatatctccaggtatgtggcgcatttacgttgtagatgtccatgggctcgagtaaccacttaacaccaggtgggctgtgagctcgtccatccatctaagcaataaatattaaaaaaagcctcgttcaaagaaggacatgtcataacgctcgggtaaaaccgtagagggggagctcattccaaaaccgcatggttcgtagcagaaaagatctctggaaacgcactgtggacgaatgcagtgtgtttctaggtagtatgcatgagctctactccaatagcggacggggtgcgaaggtaaaaacgagatgatatcctctcaaacaaaccctcagagcataataaaaataataacagattctcaaaatcctgaaatttggaaagaaatctaacaacagtattttctttgagtttcatgaggcatagacataaagctacttttacggtttaatgtcatgtttggaatgatatcttctcaaaaaatccctcagagcgctccccatgtaacatacactacaaacagagcgagaatcgagacctaaattcaattcaccacactcggagacacaagagttctccacttcagacaaaagttttgattgtctcctgcaccacgctccaagatagactctgatggcacatatatcgcgcattccccgtgctgtcatccgcatagcaacgcatgccatcaatagataataggatgaaaagcgtgggcgagagcacccagccttgtggaacgccagcgttaatggtcatggcatcgaagcagtagccatgtacgacgactgtgatggtccgtccacctaaaatggtagcgatccccttccgagtccctcgaggaattcataaattggtaacttcgacagtaccagaccctgtcggaggccttcgcgatatcaagcggacttgctctccaaggcttgagctcacctgtagcgagcgagtgtcacttcgacagaattagcgaatatctaaataactttctatatcttttgtatagaaatcttttacatatcttttatatatgctgcagagctttcgattcgaatcttaatattacatatatttaaaaggtaactaaacttcttaatacagaaaagaaaacaaaaatcaacaatgaaaggcagttagatgaagatgagaacgaaaggagaactgttgtaggccgagatgagcaaaggcaagatgaccaaagccgagacgaccaaagccgagatgaccaaagccaagatgaggaaactggcagcgacgatagtgacaaaaatagaggaaaggatactgacgataaatattccgagacaggaaccaataaatcatcagaaacgaagacaggcaagcgtgatggctcgaagagcggcgtcacagtcgaaagggaaaaatccgaatccaacaagaaaagtcgtgaatttgaaaataaagaagctgaatcttcgacctataggggtgagtactacataattaaattaaattggacacgagggaggagagagagagggaatacactttattgcacatcaaaacacagttagccttaaaaaacagttgacaaccagacactttttttattgtctagatgtgtggacgagctcacagcccacctggtgttaagtggttactagagcccatagacatccacaacgtaaatgcgccacccaccttgagatgtaagttctaaggtctcaagtatagttacaacggctgccctacccttcaaaccgacacgcaatactgcttcacggcagaaataggcagggtggtggtacccaccggcgcggactcacaagaggtcctaccaccagttaaaaccgtctggttcgtgtaccgttcgtgtgtcttttaggaaggcattgaatatactgactgctgctagtcacgtccaccctgttccatttcttttttttttcataccacctgtcgagtatcagtcattgtagacggcatgtcctgacaaagaggtttcacgccacgtcacgtattcttcacgtatattcttgtcaccgaagctctttactactcgattgacttatcatctacatcaacagataagaatcggtcagtgaacagtggctcggaacgcaagagttccggtaaagacgaggagtacagtgaacagaactccagtaataaatcctttaacgacggcgatgcatcggctgactaccaaaccaaatctaagaaggttgaaaagaattctgccagagataaaaaggaaaaggaaaaaactgacacaagaagtataattttagatagaattttgtattaaatttctgaaagcagttatttattgcaaggatttatggattattattaatattttagattctgacggaacgtacaaaacttctgagcgcgaaaaagaacaatcttctagagtgaatcaaagtaagggcagcaactctcgggattcctcggagtcagacaaatctggccgaaaagtgaataaagaaacagaaacgtactctgacaaagacgcgcagacttcagaaagtgaacgtactcaaagtaaaggtgagtgtacaatgaactacgtactacaaatgaaacatgtgaattcacagtgaattgtgtagctgcggaagtgatactctcgttcgcaggcactcacagactgcgacagaagtctgtatctagtgtacgaaagagagaatctctttttgattataaatttattttacccttttcacttaggttacgtaagtggtatctttacacatatcgagatacctagttgctagaacaacgaaaatctttctttggttgggaatttcgtttaagaattagaaaaaatagaaattggtttgaaaaaaaaaaaattttattcacagaaaaaaaaaatacagcgcccaagaataagggcaaaaagggaacctctacagaaacagatggagtcactaagaatgctagtaagcagaaggagaaggtgcctaaagatggaagtaaaagctccacgaatgatagcgaaggcaaacaaaaaaacaaagaccaatcaaagggacagaaaaataatcaagacggacaagactcttcgacgaacgaaaattccaaaaagacagatgataatgttgcaaagaaagaagaacccaataatcaaaagagagaacaaaaaggaaagacaagatgtggctctaggaaaactgagagttccaaagcgaaagaagatagaagcaagaaaagcactaccgataaggaccagcgcgacgacaaaaaagattcctccagcaaaaacatagacaagcctaaagatgggtcatcatcggataaagactcagagaaggctaagcctaacgacagatcaccatcgcataaagacacagagaaggtaaagcctaacgacagatcaccatcgcataaagacacagagaaggtaaagcctaacgacagatcaccatcggataaagacacagagaaggctaagcctaacgacagatcaccatcgcataaagacacagagaaggtaaagcctaacgacagatcaccaacgcataaagacacagagaaggtaaaacctaacgacagatcaccatcgcataaagacacagagaaggctaagcctaacgacagatcaccatcggataaagacacagagaaggctaagcctaacgacagatcaccatcgcataaagacacagagaaggtaaagcctaacgacagatcaccatcgtataaagacactgagaaggctaagcctaacgacagatcaccatcggataaagacacagagaaggctaagcctaacgacagatcaccatcgcataaagacacagagaaggctaagcataacgacagatcaccatcggataaagacacagagaaggctaagcctaacgacagatcaccatcgcataaagacacagagaaggctaagcctaacgacagatcaccatcgcataaagacacagagaaggtaaaacctaacgacagatcaccatcgcataaagacacagagaaggctaagcctaacgacagatcaccatcggataaagacacagagaaggctaagcctaacgacagatcaccatcgtataaagacactgagaaggctaagcctaacgacagatcaccatcggataaagacacagagaaggctaagcctaacgacagatcaccatcgcataaagacacagagaaggctaagcataacgacagatcaccatcggataaagacacagagaaggctaagcctaacgacagatcaccatcgcataaagacacagagaaggctaagcctaacgacagatcaccatcgcataaagacacagagaaggtaaaacctaacgacagatcaccatcgcataaagacacagagaaggctaagcctaacgacagatcaccatcggataaagacacagagaaggctaagcctaacgacagatcaccatcgcataaagacacagagaaggtaaagcctaacgacagatcacaatcgtataaagacacagagaaggctaagcctaacgacagatcaccatcggataaagacacagagaaagctaagcctaacggcagatcaccatcggataaagacacagagaaggctaagcctaacgacagatcaccatcgcataaagacacagagaaggtaaagcctaacgacagatcaccatcgcataaagacacagagaaggctaagcctaacgacagatcaccatcggatagagacacagagaaggctaagcctaacgacagatcaccatcggataaagacacagagaaagctaagcctaacggcagatcaccatcggataaagacacagagaaggtaaagcctaacgacagatcaccatcgcataaagacacagagaaggctaagcctaacgacagatcaccatcggataaagacacagagaaggctaagcctaacgacagatcaccatcggataaagacacagagaaggctaagcctaacgacagatcaccatcggataaagacacagagaaggctaagcctaacgacagatcaccatcggataaagacacagagaaagctaagcctaacggcagatcaccatcggataaagacacagagaaggtaaagcctaacgacagatcaccatcgcataaatacacagagaaggctaagcctaacgacagatcaccatcggataaagacacagagaaggctaagcctaacgacagatcaccatcggataaagacacagagaaggctaagcctaacgacagatcaccatcggataaagacacagataagacattcgacaaaaacatagacaataaaaggcccaaggacggttcatcatcggataagaacgtagagcaggaaagggagaactacaaatccgaatctagccgaaatgaatttgaaaaccaaaaatctgcacattcgagatatgagggtgggtacttttgtttaagtagcacatgcagacgcaactacgaaatgaggtggaagtttgtgtgcattataagaaacttccatttgcgctctatatgagagtcgagctcacccagtgttaagtggccatcagtccaatctatttagggtagacgacgctacaccacaaagattgtacaaggtcttctttcggggattaatttaatttaattttttgatagataacggtggtttgaaagaaaaaagctcgcaatctaagaattacggaagagacgagaagtacagtgaagagaaggagaggagttccactgggaagtttggatcgaatgactcccgagcacgttctacaaaagccgaagaggaacatgtcaggaagtcacaagaagaaacacattcagagcaacgaggtacccttcgagaaattctttgtttctaattttattgcgatcctaatcctgagtccgcacgggtaacaccgccctgcctatttctgccgtgaagcaataacgcgcttcgatttgaagggcaggtcagccgttttactgtacaaactgggaccttagaactcatgttccgagatgggtggcggcatttacgttataaatgtcaatgggctccggtaactatttaacaccggtgggaggtgagctcgtccacccatagaagcaataaaaaagaagatatattggtttgttgatattcgagaacccgacacagtcgtaattagtccgcactgtgcccttacacagtagtgtcgcacccaaaaacatttttaatcgaccactgctctgtctttacagaaaaaacaagatctgacggagtgactaaatacaacgatggtgatgagcatttcgattcagacgatacagagaagactaagcctaatggcagatcaccatcgcataaagacacagagaaggctaagcctaacgacagatcatcctcggataaagacacagagaagacatttgacaaaaacatagacaataaaaggcccaaggacggttcatcatcggataagaacgtagagcaggaaagggagaactacaaatccgaatctagccgaaatgaatttgaaaaccaaaaatctgcacattcgagatatgagggtgggtacttttgtttaagtagcacatgcagacgcaactacgaaatgaggtggaagtttgtgtgcattataagaaacttccatttgcgctctatatgagagtcgagctcacccagtgttaagtggccatcagtccaatctatttagggtagacgacgctacaccacaaagattgtacaaggtcttctttcggggattaatttaatttaattttttgatagataacggtggtttgaaagaaaaaagctcgcaatctaagaattacggaagagacgagaagtacagtgaagagaaggagaggagttccactgggaagtctggatcgaatgactcccgagcacgttctacaaaagccgaagaggaacatgtcaggaagtcacaagaagaaacacattcagagcaacgaggtacccttcgagaaattctttgtttctaattttattgcgatcctagtcctgagtgcgcacgggtaccaccgccctgcctatttctgccgtgaagcaataacgcgcttcgatttgaagggcaggtcagccgttttattgtacaaactgggaccttagaactcatgttccgagatgggtggcggcatttatgttataaatgtcaatgggctccggtaactatttaacaccggtgggaggtgagctcgtccacccatagaagcaataaaaaagaagatatattggtttgttgatattcgagaacccgacacagtcgtaattagtccgcactgtgcccttacacagtaacgcaccgaaaaatattttaatcgaccactgctctgtctttacaggaagaacaagatctgacggagcaactacgtccaacgataatgataagcaatacgattcagacgacaagaacaacagtagcaccaaacataagaaaacggtcatgagatctgagcaatcagattcgtctcaaaatgaaaattctacctccgaatccaaaaagttcgcaaaaactgatggctccaacaaatatgaagcggaatcaagttcgcataaacaacaagaagcgcgcaaacagagcaatagagtagtagagaagagcactgatggggacaatgaggaatcctacaggagcgaaagcagcagcagcagcagcagcagttctagtagtagtcgaagcagttcctcaagcacctacaccggctcccatgatgatagctccgaggaatgacgaatgggaacatcaactttaacaaaccttgcattaccttatggctttgtgtagttttaagtttgtgaatgcatttaaaaattgtgatggccgttcctcgtttggttgactcatttttaacagtttgtagctagttaaaataagtcgccgcgtgtatcattggctatcacacaaaaaatataactttttaaataaaaacctaagcatac

SEQ ID NO: 3 - DNA, 6651 bp, Bombyx mori
taactgtttattcataaagggaatatttctccaacaaattagtataaatagccggcctttgggcgtttacagacagagcaatcgaagcttcgaagatgaattgtaaagttgctctattcctgatagtggtaagtcatttctaagatattctccgataaaggatccaaaaacaccgcgaagacagtcgtttggattcgctggtacttaaaaagcttggcattcgctgtatcccaggggaacgctttgaacaattcagttcggaggttcaattcttattcgcaccatttgccgacaaacagggacgctactgctgcgcctgttctcggtaaatttccacaaattatattggcttttaaattggttttatggttaattgaacacacaagcagctgccgaaacggtgttttctatatttattccgcgtattaaagccaataaagttcttcgaatttcatcttgtgacgatacaattaacaatcgattccacaacaatagcaggtaacatagcaagaagagagcaagcatttaagttaagaatgtcgtggataaaaatcgcgcaaatgttttcaaccgcggctggagctttatcttcaactttgctccttagagcttagagctccgtcacacttcatttgtaagggccaaagaccaagtagtgatggggacattaaagaagcagagcgtcctcgagttataccatcattgtcatcatcatccgaacgccgtcgtttactcctgcacataagcccaatccacgccacttatatatcacaagtgaatactcctcaactcagccgaagagttcgattcgtataaataagcatgtcccgtttagcgatgcggatctcagaggcaattcgttcagtctcccatcgtctttgcggcttggtatccacatgttcttttctcactggattctcctcctgattcctgttcaactgaatattagcgctggggcgtagcatcgtcgcattctgaactataaagacgtggttttagtattactttcgctggtgctgcgcaaaaatgtttattttacataccggtgagatgaataaatttaattgttctaatgtattaatgaggatcagtgggatgcggtatttaaagctctacagtaaccatgtcgtgttatttgtcattcgtatacgcttcggatatgcttggtacacagtgccatcagtgtttgactggttggttgggtcccaatcatttacatggatggtgcttttaaggcttttctgagtaataaaaaagagcgagactttaacagttcggtcctgctctgctcgcatccagcagatcctcgcgagtctcaataggccatcggtccacctatccatgccacgcctttcagcttggtggaggtctatgtccagtattgtaagtctctgagctaatagataaaaagataaataattcgttgtttctttttcaggctattgtagccgtccaggctctgccttgccgtaagtaatgttttaactgttacaaacaaaaacgtttgcgtccacacgcaatgctaaattaagaatttttggagtttactcctttagaatcgatttacatatataggcccggggtatgaaaattatggggaccaaaatcgtactagcatgtcacacgaaatgcgttatgcgcctgattggctcctgattgcgtgatgcgtgcgcgcgccaatcaaaatcgtactagcatgtcacacgaaatgcgttatgcgcctgattggctcctgattgcgtgatgcgtgcgcgcgccaatcaggagccaacgcgcggccgcgttattgcaggtgacgccgggctgctgcgccggcagtccgccacaaagcctcgtactgatcgcgcgtttctctcattattgtattttcatatatttgttagtcgtttgttttgtgtctcgttaatttgttaataatctgtagtgtatcgtgttgtcgtaatatagaactatttctcgtattgtttcgtttaattttttatatccagtaaactccagtcgtgcgtcggagcggacacacacacttattttttttcctgatatgccgtacaaacgtggttatacttcggcgattaggtgtgccggaacttatagcatatttatcacctaaagacagtcctatgaccacctatggaccaagataatatcatgggcgaactcttttaattattaaaatgctcgttgaatgctattttttacttcaaaaaagaaggaggttctcaattcgactatatgttttttttatgtttgtttttttataacttttgactgggaaaatcgattttgatgattctttttttattagaaagctgatgcttcctgcgtggtcccatttcaatttagtacagttctgttaatggtatccatgagaaaaccatgtaataagtcttaaatttgcattaagtacgtgcacgacaaatagatgaataactcaataactcaatatcacgccaaccgatttcgatgattattgttttagtgtacattcatttgttttgttatatcttttttttttctatttgaagtcggtttttgttcaagcatgtctatttacaattatttatagtcttgtggaatatttccagaggaatctagaccgagacgctgcgggtacaggtatggctgcggaggcggtcgaggcaggggctctggtgtgagacgtctcgattcaggagcagatgacaaccaagggcgatcatgtctcgtcggtggaagtgcagaatcaaatgacgaggactccgatgatcgtttatcagtaatcaataatactgaaggccacgctaccagcagcagcttcgatgagcaaagttctagcgctagacaaagcagtagctcgtaccagagccagtcatacaacaaagattcttcaagtaccaatgaaagcagtgatggaggctctggttcgggcagaaccggttcagcaggagaaaatggtgagaattccgacgacagcagtggtgcgacaaaaggaaatagcagtaaatcttccagcagttctcaaggtcaaagcgcaagtagtagcagcagtgatgaaaaatcatctcagagcagcagtaatagtagcaacaacagtaaatcaagcagccaatcttccagtagccaaaacagttctggttctaagggctcgggatcagaagaaagcagtaatggaggctctggttcgggaagaaccggttcagcgggaggaactgatgaggattccgacgacagcagtggtgcgacaaaaggaaatagcagtaagtcttccagcagttctcaaggtcaaagcgcaagcagtagcagcagtgacgaaaactcatctcagagcagcagtaatagtagcaacaacagtaaatcaagcagccaatcttccagtggccaaaacagttctggttctaagggctcgggatccgaagaaagcagtaatggaggctctggttcgggaagaaacggttcagtgggaggaactgatgaggattccgacgacagcagtggtgcgacaaaaggaaatagcagtaaatcttccagcagctctcaaggtcaaagcgcaagcagtagcagcagtgatgaaaaatcatctcagagcagcagtaatagtagcaacaacagtaaatcaagcagccaatcttccagtggccaaaacagttctggttctaagggctcgggatcagaagaaagcagtaatggaggctctggttcgggaagaaacggttcagcgggaggaactgatgaggattccgacgacagcagtggtgcgacaaaaggaaatagcagtaagtcttccagcagttctcaaggtcaaagcgcaagcagtagcagcagtgatgaaaaatcatctcagagcagcagtaatagtagcaacaacagtaaatcaagcagccaatcttccagtggccaaaacagttctggttctaagggctcgggatcagaagaaagcagtaatggaggctctggttcgggaagaaacggttcagcgggaggaactgatgaggattccgacgacagcagtggtgcgacaaaaggaaatagcagtaagtcttccagcagttctcaaggtcaaagcgcaagcagtagcagcagtgacgaaaaatcatctcagagcagcagtaatagtagcaacaacagtaaatcaagcagccaatcttccagtggccaaaacagttctggttctaagggctcgggatcagaagaaagcagtaatggaggctctggttcgggaagaaccggttcagcgggagaaactgatgaggattccgacgacagcagtggtgcgacaaaaggaaatagcagtaagtcttccagcagttctcaaggtaaaagtgcaagcagtagcagcagtgacgaaaaatcatctcagagtagcagtaatagtagtaacaacagtaaatcaagcagtcaatcttcgagtagcaacaatagttctggttctaagggctcgggatcagaagaaagcagtaatggaggctctggttcgggaagaaccggttcagcgggaggaagtgatgaggattccgacgacagcagtggtgcgacaaaaggaaatagcagtaagtcttccagcagctctcaaggtcaaagcgcaagcagtagcagcagtgacgaaaaatcatcacagagcaacagtaatagtagcaataacagtaaatcaagtagccaatcttcgagtagcaacaacagttctggttctaagggctcgggatcagaagaaagcagtaatggaggctctggttcgggaagaaccggttcagcgggaggaactgatgaggattccgatgacagcagtggtgcgacaaaaggaaatagcagtaagtcttccagcagttctcaaggtaaaagcgcaagcagtagcagcagtgacgaaaaatcatctcagagcagcagtaatagtagtaataacagtaaatcaagcagccaatcgtccagtagcaagaacagttctggttctaagggctcgggatcagaagaaagcagtaatggaggctctggttcgggaagaaccggttcagcgggaggaactgatgaggattccgacgacagcagtggtgcgacaaaaggaaatagcagtaagtcttccagcagttctcaaggtaaaagcgcaagcagtagcagcagtgacgaaaaatcatctcagagtaacagtaatagtagtaacaacagtaaatcaagcagtcaatcttcgagtagtaagaacagttctggttctaagggctcgggatcagaagaaagcagtaatggaggctctggttcgggaagaaccggttcagcgggaggaactgatgaggattccgacgacagcagtggtgcgacaaaaggaaatagcagtaagtcttccagcagttctcaaggtaaaagcgcaagcagtagcagcagtaacgaaaaatcatctcagagtagcagtaatagtagtaacaacagtaaatcaagcagtcaatcttcgagtagcaagaacagttctggttctaagggctctggatcaagtgaaagtggtgataaaaagtccagttctcgaggaagttctggtgacaactcagacgatgaccaaactgattcagccagatcaaatagtaagcgttccacaagctctgatgcgtccactaaaaaaagttcgtctagaaagagctccaaccaccgtagtagcagaagtcagcaagctcatagtagcagcagtaaacaagcccaaagcagcagtagtcaacaagcccaaaatagcagaagtcagcaagctcatagtagcagaagtcagcaagctcatagtagcagcagtaaacaagcccaaagcagcagtagtaaacaagcccaaagtagcagcagtaaacaagcccaaagcagcagtagtaaacaagcccaaagtagcagtagtcaacaagcccaaagtagcagaagtcagcaagctcatagtagcagaagtcagcaagctcatagtagcagcagtaaacaagcccaaagcagcagtagtaaacaagcccaaagtagcagcagtaaacaagcccaaagcagcagtagtaaacaagcccaaagcagcagtagtcaacaagcccaaagtagcagaagtcagcaagctcatagtagcagaagtgagacagatagtaagagcagtaatagtggtggccattctaatcatagcagtagaactgaacaaaagagcagcgcaaaagcaatttcaagcagtgaacagtcacagaatttctcaagcagttcacagaaatctgccgaggccgctgatggatctgaaagcacccagtcagcaagtgaatactcgagcagccaatcaaaggccagtagcagttttagcgcttcgagtgccagtgaaagttcttcattaagcagcgacgtgaacttcgaagagaagaccgactaaaacggccaacgatttatatgtttcatgttaatagcgataaaggtaatattatttgggcaccgttccgcacctcgtttaattaccatagcgggcacgatgcgatggtttctgtaacgattcgtcaattatttcgttaatagaactacatataagacttcctttatcgatattaatgtgagctgaaaacgcaaaacacaatatacgatagcttttattacatttagaacgcaaaattcatattggaagaacattaacatgtttactgtaaaactatttaacttttggatataacataaataaattacgaacattaa

SEQ ID NO: 4 - DNA, 21 bp, Bombyx mori
tataattacgagttaaatact

SEQ ID NO: 5 - DNA, 21 bp, Bombyx mori
agtggagaccgcgtgccggca

SEQ ID NO: 6 - DNA, 21 bp, Bombyx mori
ttaatggggtggcgtagcata

SEQ ID NO: 7 - DNA, 21 bp, Bombyx mori
ggtctaaatataaatggagct

SEQ ID NO: 8 - DNA, 21 bp, Bombyx mori
aggccaataagggctaataca

SEQ ID NO: 9 - DNA, 21 bp, Bombyx mori
gcggcgtctagaactagcgga

SEQ ID NO: 10 - DNA, 21 bp, Bombyx mori
gtctacaagagatgaaaagct

SEQ ID NO: 11 - DNA, 21 bp, Bombyx mori
tggatacagttccaacagtcg

SEQ ID NO: 12 - DNA, 21 bp, Bombyx mori
aatcagtgttactggaactgc

SEQ ID NO: 13 - DNA, 21 bp, Bombyx mori
aaagtatatagggaaaatagg

SEQ ID NO: 14 - DNA, 21 bp, Bombyx mori
aaaatctcaaagacaatgttg

SEQ ID NO: 15 - DNA, 21 bp, Bombyx mori
gaaaaagaaggatacaagtct

SEQ ID NO: 16 - DNA, 21 bp, Bombyx mori
gttcgctcggcactaataccg

SEQ ID NO: 17 - DNA, 21 bp, Bombyx mori
aagaagcaggataattcaaaa

SEQ ID NO: 18 - DNA, 21 bp, Bombyx mori
ttggttcctgtctcggaatat

SEQ ID NO: 19 - DNA, 21 bp, Bombyx mori
tagagtgaatcaaagtaaggg

SEQ ID NO: 20 - DNA, 21 bp, Bombyx mori
gacgaacgaaaattccaaaaa

SEQ ID NO: 21 - DNA, 21 bp, Bombyx mori
tgcgatggtgatctgtcgtta

SEQ ID NO: 22 - DNA, 21 bp, Bombyx mori
aaagaaaaaagctcgcaatct

SEQ ID NO: 23 - DNA, 21 bp, Bombyx mori
gattcagacgatacagagaag

SEQ ID NO: 24 - DNA, 21 bp, Bombyx mori
agatccatcagcggcctcggc

SEQ ID NO: 25 - DNA, 21 bp, Bombyx mori
tgctactacggtggttggagc

SEQ ID NO: 26 - DNA, 21 bp, Bombyx mori
ctgttgttactactattactg

SEQ ID NO: 27 - DNA, 21 bp, Bombyx mori
cagcagttctcaaggtaaaag

SEQ ID NO: 28 - DNA, 21 bp, Bombyx mori
ccgaaccagagcctccattac

SEQ ID NO: 29 - DNA, 21 bp, Bombyx mori
ctgttgttgctactattactg

SEQ ID NO: 30 - DNA, 21 bp, Bombyx mori
cagtggccaaaacagttctgg

SEQ ID NO: 31 - DNA, 21 bp, Bombyx mori
agtattattgattactgataa

SEQ ID NO: 32 - DNA, 21 bp, Bombyx mori
ccagtcaaaagttataaaaaa

SEQ ID NO: 33 - DNA, 21 bp, Bombyx mori
tgtgcaggagtaaacgacggc

SEQ ID NO: 34 - DNA, 7308 bp, Unknown
  repeating motifs of MaSp2 and flagelliform
cccccaggcggtgcaggtccaggcggtgcgggtccaggcggagcaggcccaggcggagcaggtccaggcggagcgggtccaggtggtgctggccccggtggagccggtcctggcggtgctggacctggaggcccatcaggtcctggttctgctgccgcagctgctgccgctgccggcccaggtggagccggtccgggcggtgctggacccggaggcgccggccctggtggagctggtccaggcggtgccggaccgggaggcgctggccccggtggagccggtcctggcggtgctggacccggaggccctagcggaccaggctccgctgctgcagccgcagctgcagctggcccaggtggagccggtccgggcggtgctggacccggaggcgccggccctggtggagctggtccaggcggtgccggaccgggaggcgctggccccggtggagccggtcctggcggtgctggaccgggaggcccctcgggtcctggaagtgctgccgcagcagcagctgcagcaggcccaggtggagccggtccgggcggtgctggacccggaggcgccggccctggtggagctggtccaggcggtgccggaccgggaggcgctggccccggtggagccggtcctggcggtgctggtcctggtggtccttcaggccccggttctgctgccgcagcagccgctgcagcgggcccaggtggagccggtccgggcggtgctggacccggaggcgccggccctggtggagctggtccaggcggtgccggaccgggaggcgctggccccggtggagccggtcctggcggtgctggacctggaggccccagcggaccgggctctgcagctgccgctgctgctgctgctggcccaggtggagccggtccgggcggtgctggacccggaggcgccggccctggtggagctggtccaggcggtgccggaccgggaggcgctggccccggtggagccggtcctggcggtgctggacccggaggcccctcgggtccaggaagtgctgccgcagcggcagctgcagccggcccaggtggagccggtccgggcggtgctggacccggaggcgccggccctggtggagctggtccaggcggtgccggaccgggaggcgctggccccggtggagccggtcctggcggtgctggaccgggaggcccttcaggccctggttctgctgccgccgctgcagctgccgcaggcccaggtggagccggtccgggcggtgctggacccggaggcgccggccctggtggagctggtccaggcggtgccggaccgggaggcgctggccccggtggagccggtcctggcggtgctggtcctggtggtccgagcggacccggctctgcagctgcggctgccgctgcagctggcccaggtggagccggtccgggcggtgctggacccggaggcgccggccctggtggagctggtccaggcggtgccggaccgggaggcgctggccccggtggagccggtcctggcggtgctggacctggaggcccttctggtccgggaagtgctgccgcagccgcagctgccgccggcccaggtggagccggtccgggcggtgctggacccggaggcgccggccctggtggagctggtccaggcggtgccggaccgggaggcgctggccccggtggagccggtcctggcggtgctggacccggaggcccatcaggcccaggttctgctgccgcagcggccgctgccgccggcccaggtggagccggtccgggcggtgctggacccggaggcgccggccctggtggagctggtccaggcggtgccggaccgggaggcgctggccccggtggagccggtcctggcggtgctggaccgggaggcccaagcggacctggctccgctgctgccgccgcagctgcggctggcccaggtggagccggtccgggcggtgctggacccggaggcgccggccctggtggagctggtccaggcggtgccggaccgggaggcgctggccccggtggagccggtcctggcggtgctggtcctggtggtccttctggtcccggaagtgctgccgccgctgccgctgcggcaggcccaggtggagccggtccgggcggtgctggacccggaggcgccggccctggtggagctggtccaggcggtgccggaccgggaggcgctggccccggtggagccggtcctggcggtgctggacctggaggcccgtctggtcctggttcagctgccgcagcagcagcagcggcgggcccaggtggagccggtccgggcggtgctggacccggaggcgccggccctggtggagctggtccaggcggtgccggaccgggaggcgctggccccggtggagccggtcctggcggtgctggacccggaggcccgtctggaccaggctctgctgccgcagcagcggctgcggccggcccaggtggagccggtccgggcggtgctggacccggaggcgccggccctggtggagctggtccaggcggtgccggaccgggaggcgctggccccggtggagccggtcctggcggtgctggaccgggaggcccgtcaggtcctggatctgctgccgcagcagctgctgcagccggcccaggtggagccggtccgggcggtgctggacccggaggcgccggccctggtggagctggtccaggcggtgccggaccgggaggcgctggccccggtggagccggtcctggcggtgctggtcctggtggtcctagcggccccggttcagcagccgctgccgcagcagctgcgggcccaggtggagccggtccgggcggtgctggacccggaggcgccggccctggtggagctggtccaggcggtgccggaccgggaggcgctggccccggtggagccggtcctggcggtgctggacctggaggcccctctggaccgggctctgcggctgcagctgctgctgcagctggcccaggtggagccggtccgggcggtgctggacccggaggcgccggccctggtggagctggtccaggcggtgccggaccgggaggcgctggccccggtggagccggtcctggcggtgctggacccggaggccccagcggtccaggatctgcagccgctgccgcagcggcggcgggcccaggtggagccggtccgggcggtgctggacccggaggcgccggccctggtggagctggtccaggcggtgccggaccgggaggcgctggccccggtggagccggtcctggcggtgctggaccgggaggccccagcggccctggttcagcagctgcagccgcagctgctgcaggcccaggtggagccggtccgggcggtgctggacccggaggcgccggccctggtggagctggtccaggcggtgccggaccgggaggcgctggccccggtggagccggtcctggcggtgctggtcctggtggtccaagcggacccggctctgcggctgccgctgccgctgcagctggcccaggtggagccggtccgggcggtgctggacccggaggcgccggccctggtggagctggtccaggcggtgccggaccgggaggcgctggccccggtggagccggtcctggcggtgctggacctggaggcccctctggtccgggatctgcagctgcagccgccgctgctgccggcccaggtggagccggtccgggcggtgctggacccggaggcgccggccctggtggagctggtccaggcggtgccggaccgggaggcgctggccccggtggagccggtcctggcggtgctggacccggaggccccagtggcccaggttcagcagctgccgctgcagctgctgccggcccaggtggagccggtccgggcggtgctggacccggaggcgccggccctggtggagctggtccaggcggtgccggaccgggaggcgctggccccggtggagccggtcctggcggtgctggaccgggaggccccagtggacctggctccgctgccgcagccgcagcagcagccggcccaggtggagccggtccgggcggtgctggacccggaggcgccggccctggtggagctggtccaggcggtgccggaccgggaggcgctggccccggtggagccggtcctggcggtgctggtcctggtggtccttctggtcccggatctgctgccgcagcggcggctgctgcgggcccaggtggagccggtccgggcggtgctggacccggaggcgccggccctggtggagctggtccaggcggtgccggaccgggaggcgctggccccggtggagccggtcctggcggtgctggacctggaggccccagtggtcctggttccgcagccgctgccgcagccgccgccggcccaggtggagccggtccgggcggtgctggacccggaggcgccggccctggtggagctggtccaggcggtgccggaccgggaggcgctggccccggtggagccggtcctggcggtgctggacccggaggccccagcggaccaggctctgcagccgctgccgccgctgctgccggcccaggtggagccggtccgggcggtgctggacccggaggcgccggccctggtggagctggtccaggcggtgccggaccgggaggcgctggccccggtggagccggtcctggcggtgctggaccgggaggccccagcggtcctggatcagcagccgctgccgccgcggcagcgggcccaggtggagccggtccgggcggtgctggacccggaggcgccggccctggtggagctggtccaggcggtgccggaccgggaggcgctggccccggtggagccggtcctggcggtgctggtcctggtggtcctagtggccccggttcagccgctgcagccgcagcagccgcgggcccaggtggagccggtccgggcggtgctggacccggaggcgccggccctggtggagctggtccaggcggtgccggaccgggaggcgctggccccggtggagccggtcctggcggtgctggacctggaggccccagcggaccgggctctgctgctgccgctgcagctgctgctggcccaggtggagccggtccgggcggtgctggacccggaggcgccggccctggtggagctggtccaggcggtgccggaccgggaggcgctggccccggtggagccggtcctggcggtgctggacccggaggccccagcggtccaggatctgctgctgcagctgcagctgcagctggcccaggtggagccggaccgggcggtgctggtcccggaggcgccggacctggtggagctggtccaggcggtgccggaccgggaggcgctggtcccggtggagccggacctggcggtgctggtccgggaggcccctctggccctggttcagctgccgcggctgctgccgctgccggaccaggtggagccggtccgggcggtgctggacccggaggcgccggtcctggtggagctggaccaggcggtgccggtccgggaggcgctggacccggtggagccggtcctggcggtgctggtcctggtggtccttctggacccggctctgctgctgctgctgctgccgctgccggtccaggtggagctggaccgggcggtgctggtcccggaggcgctggacctggtggagctggtccaggcggtgctggaccgggaggcgctggtcccggtggagctggacctggcggtgctggtcctggaggcccatctggtccgggatctgcggctgcagctgctgctgccgctggaccaggtggagctggtccgggcggtgctggacccggaggcgctggtcctggtggagctggaccaggcggtgctggtccgggaggcgctggacccggtggagctggtcctggcggtgctggacccggaggccccagcggcccaggttcagcggctgccgctgcggctgcagctggtccaggtggagctggaccgggcggtgctggtcccggaggcgctggacctggtggagctggtccaggcggtgctggaccgggaggcgctggtcccggtggagctggacctggcggtgctggtccgggaggccctagcggacctggctctgcagctgcagctgccgctgccgcaggaccaggtggagctggtccgggcggtgctggacccggaggcgctggtcctggtggagctggaccaggcggtgctggtccgggaggcgctggacccggtggagctggtcctggcggtgctggtcctggtggtccttctggtcccggatctgcagctgcagctgcggctgctgctggtccaggtggagctggaccgggcggtgctggtcccggaggcgctggacctggtggagctggtccaggcggtgctggaccgggaggcgctggtcctggtggagctggaccaggcggtgctggtcctggaggccctagtggtcctggttctgcagctgcagctgctgctgctgctggaccgggtggagctggtcctggcggtgctggtcctggtggtgctggtccgggtggagctggacctggcggtgctggtcctggtggtgctggaccgggtggagctggtcctggcggtgctggacccggaggccccagcggaccaggctctgcggctgcggctgctgctgccgcaggtccaggtggagctggaccgggcggtgctggtcctggaggcgctggaccaggtggagctggtccgggcggtgctggacctggaggcgctggtccaggtggagctggaccgggcggtgctggtccgggaggcccatctggtcctggatctgcagctgcggctgctgctgctgctggacctggtggagctggtccaggcggtgctggaccgggaggcgctggtcctggtggagctggaccaggcggtgctggtccgggaggcgctggacctggtggagctggtccaggcggtgctggtcctggtggtcctagcggccccggtagcgctgctgccgctgccgctgccgctggtccgggtggagctggacctggcggtgctggtcctggtggtgctggaccgggtggagctggtccaggcggtgctggacctggaggcgctggtccaggtggagctggacctggcggtgctggtcctggaggcccgtctggaccgggctcagcagctgcagctgccgcagcagccggaccaggtggagctggtcctggcggtgctggtcctggtggtgctggtcctggtggagctggtccaggcggtgctggtcctggaggcgctggtcctggtggagctggtcctggaggagcaggtccaggtggtccctcaggtccaggttcggcggcggcggcggcagcagcagcg

SEQ ID NO: 35 - DNA, 3156 bp, Caerostris darwini
gccgggggtgcgggcggacgaggaggattaggggggcagggcgggggtcagggcgctggaggagccggacagggaggctatggaagcggcttgggaggcttagggggcggagccgctgcagctgcagccgctgccggcggtgcaggcggcctgggcgggcaagggggtggccaaggcgctggccaaggcggctacggatcaggtcagggtggccaaggcgccggaagcgcggctgccgcggccgctgcggggggcgcaggcggtaggggggggctagggggtcagggcggtgggcagggtgccggtggcgccggtcaggggggatacggatctggactggggggcttgggcgggggggcagcggcggccgcggccgcagccggaggagcaggagggctcggtggacaaggcggagggcagggcgctggtcaaggcggctatggttccggccaaggagggcaaggagccggctcggctgccgcagcagcggccgcgggtggtcgaggaggatatggtgggcaagggggcgggcaaggagcggggggtgctgggcaaggtggttacgggagtgggcttggtggactgggtggtggcgcagcagccgcggcggcggcggcgggaggcgctggcggcctcggaggacagggaggaggtcaaggcgctgggcagggaggctacggtagcggtcagggcggtcagggcgccgggtccgctgcggccgcggccgctgccggaggagccggcggccttggcgggtatgggggccaggggggaggacagggggcgggaggagctggccaggggggttatgggtcaggtttagggggccttggcggtggagcagccgcggccgctgcagcagcgggtggcgcgggtgggctggtcggtcagggcggtgggcagggcgcagggcagggagggtatggtagtgggcaaggagggcaaggcgctgggtcagcagcggctgcagccgcagcgggaggtgctgggggcagaggcggtcttggtgggcagggcgggggccagggcgccggcggggcagggcaagggggttatggaagcgggctcgggggtttgggtgggggcgcgtcagctgcggcagctgctgcggggggtgccggagggctcggaggacaggggggtggccaaggtgcgggccaggggggatacggaagcggccaaggggggcagggagctgggtctgccgcagctgctgcggcagcggggggatcaggcggattggggggacagggcggatacggtgggcaggggggctacggcggtggctatggcgggcaacagcaggggggggggcagggggctgggcaagggggttatggatcaggccagggaggtcagggcgcgggttccgcggcagctgctgcagccgcggggggacgtggaggatacggcgggcagggcggaggacagggtgcgggtggggccggacaaggcggatatggatctggcttgggtggactagggggcggagccgcggcagctgcggggggagcgggaggactcggaggacagggagggggccaaggggctggacaaggaggctacggatcggggcagggcgggcagggcgctggatcagctgcggcggccgctgctgctgggggaagaggcggttatggtgggcaggggggcgggcagggcgctgggggcgctggccaagggggctatggctccggtctggggggcctcggaggtggagctgccgctgctgcagcagcggccggcggtgcgggtggtctcgcagggcaaggtggtggacagggagccggccaaggagggtatggatcaggtcagggaggccagggtgctggctctgcagcagcggctgccgcggccggaggcgccgggggacgcggtggctatgggggtcaagggggacagggtgctggaggtgctggccaaggcggatatggctctggccttggcggtctaggggggggggctgccgccgcggcagcagcggcggggggtgcagggggcctgggcgggcagggtgggggccagggtgctggtcagggtggctatggttcgggtcaggggggacaaggcgcaggctccgcagcggcagcggccgctgcaggaggcgcggggggacaggggggatacggtggacaagggggccagggggcgggtggggctggtcaaggcggttacggttcgggtcttggaggtgtcgggggtggcgctgcggctggaggcgcaggcggattgggaggtcaggggggcggtcagggcgcaggtcagggtgggtatggctcagggcagggaggccagggcgcaggcagtgcggcggcagcgggacgcggcgggtacggcgggcgtgggggcgcaggcagctcttcggcagcagctgctgcaggttcggggggtgatggtagcggcagtggggggtacggtgggcgcgggggtcaaggaggtgatggcgcgtcttcggctgccgcagcggctggaagcgggggtgatggatccggatcaggaggttacggcgggaggggcggacgaggcgggcagggtggggcggggagcagtagtgccgccgcggcggcagggagcggaggagatggatcgggaagcgggggttacggagggcgaggaggtaggggcggggctggatcttcgagtgccgcgagcgcagctgcgggaggtgaggatggattcggtcgtggcgggtacggtggtagggggggagcagggtcctcttcggccgctgcagccgcaggcagcggcggagacgggtccggttcgggggggtatggcgggagaggtggtcagggcggagcgggtagtagttcagcagctgctgctggttctgggggcgatgggtacgggtctggcggatatggtgggcgaggagggcaaggaggggcaggatcttctagtgcagccagcgcagctggaagtggaggtgatgggttcggatcgggtttttacgggggtagagggggggaaggtggcgccggaagctcttccgcggcggcagcagctggcagtggaggggacggctacgggtccgggggatacggcggacgcggaggacaggggggtgcgggaggcgcttcggcttcagccgtcgcagcgggaggcggccgtgggcagggggggtatggcgggaggggtggtcaagggggagcagga

SEQ ID NO: 36 - DNA, 8484 bp, Latrodectus hesperus
ggacaagggggcgcgggacctggtggtgcaggtgccgcggcagcagcggcggccgcggctggcggggccgggcaaggcggtcaagggggatacggccaaggtggttacggtcaagggggggcgggtcaaggcggcagcggtgccgcggcagcggcagccgcagcagccggcgggactggacaaggcggagcgggtcagggtggggccggagcagcagcagctgctgccgcggcggccggcggagcgggccaaggagggcaaggcggctatggccaaggcggctatggacagggaggaacgggccaaggtggagcgggtgctgctgcagctgccgcggcagcggggggagcgggacaaggaggccaaggtggctatggccaggggggatatggccagggaggatacggtcagggtggttccggcgcggctgccgccgcggcagcggcggccggaggcgcgggtcaaggtggtcaaggcggatacggacaggggggctatggccaagggggcgcagggcaagggggcgcaggagcggccgctgctgctgcggcggctgcgggcggtgctgggcagggaggatatggacgcggtggtgcaggccaaggaggtgctgcggcagccgctgccgcagcggccggtgcaggacaaggagggtatggcggtcaaggtgcggggcaaggaggaagcggagccgccgctgcagcggcagcggcaggtggcgcaggccagggaggtcaaggtggctatggacagggaggttacggccagggaggttcaggcgctgctgcagctgctgcagccgccgggggcgctggccagggaggtcaaggcggctatggccaagggggctacggccaaggtggggctggtcaagggggcgcaggagccgctgcagctgcagcggcggcggggggagctgggcaggggggtcaaggcggttatggccaaggaggatacggtcaaggaggagcgggacaaggtggtgcgggggccgcggccgccgcagccgctgcggggggagccggtcaaggtgggcagggcgggtatggtcagggcggttatggtcaaggaggggccggtcagggcggtgctggcgctgccgcggctgctgctgcagccgggggggccggacaaggggggcagggaggttatgggcaggggggatacggtcagggtggagcgggacagggtggtgccgcggccgcggcggcggcggccgctggtggtgcgggtcaagggggctatggccgtggaggtgcgggacagggcggggccgctgctgcagcaggtgctggacagggtggatatggcggacagggcgcaggccaaggaggtgcaggagcagctgcggctgcggcggccgccggcggtgcgggacagggtggccaggggggctatggaaggggtgggtacggtcaaggaggagcgggacaaggcggggcgggcgcagcagcggccgccgccgcggcgggcggcgcggggcagggtgggcaaggcggctacggccaaggagggtacggacagggtggcgctggccaaggcggtgccgcggctgctgccgctgcggcgggaggtgcaggtcagggcggatacggtagggggggcgctggtcaagggggggctgcggccgccgcggctgctgccgcgggtgctggccaaggaggctacggcggtcaaggagcgggccagggcggagctggtgccgctgcggccgcggccgctgctggaggcgcaggccaaggagggcagggagactacggcagaggaggatatgggcaaggaggggccgggcaggggggcgcgggcgccgcggcggccgccgctgctgctggcggtgcaggtcagggtgggcagggaggttatgggcagggcggatacggccagggtggggccgggcaggggggtgcagcagcggcagcttctgccgctgctgcaggcggagctggacagggaggatacgggagaggtggcgcaggccaaggcggagctgccgcggcggccggggcgggacaaggggggtacgggggtcagggcgctggccaaggcggcgccggagctgccgcggcagctgcggctgccggaggagcgggacagggaggccaaggcggttacggacgtggaggttatggtcaagggggagcgggacaggggggggcgggcgcagccgctgcagccactgcagcagggggtgcgggtcaaggaggacagggaggatatggtcagggtggttatggtcaaggcggagctggccagggcggggcggctgctgccgcagcggctgcagcgggaggcgccgggcaggggggctatggccgtgggggggctggacaaggaggcgccgcagccgcagccgccgctgccgcaggagccggtcagggcggctacggcgggcaaggagctggacaggggggagcgggagcagctgcagcagcggctgggggagccggacaaggtggacaaggcggttatggaaggggggggtacggacaaggcggcgcgggccaggggggcgctggagcggcagctgcggcagccgcggcgggtggagcgggtcagggtggacaaggtggttacggccaaggcggatatggtcaaggaggtgccggacaggggggtgcggcagccgcagccgcggcggcagcgggcggggcaggtcagggcggctacgggaggggtggcgctggtcaaggaggcgcagctgcggccgcaggcgctgggcagggcgggtatgggggacagggtgccgggcaaggtggtgctggcgccgcggcagcggcatcgaggggagctggtcagggagggcagggagggtacggacgcggtggttatggccagggaggcgccgggcaagggggagcaggagccgccgcagctgcggccgccgctggtggcgccggtcaggggggacagggtggttatgggcaaggcgggtacgggcaaggtggtgcgggtcaagggggtgctgcagccgcggcggccgctgcggggggggcgggacagggcggctacggtcgcggcggggcaggacagggcggtgccgcagccgctgccggtgccggccaagggggttatgggggtcagggcgcgggtcaggggggtgctggggccgctgcagcggcagcggccgcagggggcgcaggccagggggggcaggggggctatggcaggggcggatacggtcagggaggggctgggcagggtggagcaggggcggcagccgcagcagcagctgccggtggtgcagggcaggggggccaaggtggatacggtcagggcggctatgggcagggcggcgctgggcaaggaggtgcggcggcggccgctgcggctgcagctggtggtgccgggcaggggggctatggcagggggggcgcgggacaaggcggggctgctgcagctgccgcagccgcggcagggagcggccaaggcggttacggcggccagggagcgggtcaggggggagctggcgccgccgctgcagcggcggctgcgggtggtgctggccaaggtggtcagggtgggtacggccgcggcggatacggccagggtggggcgggccaggggggagcgggtgccgcagccgcggcagcggcagctggaggcgcgggtcaaggcggacagggtggttatggacagggtgggtatggacagggtggggcaggacaaggaggagcggcagctgcggctgcagcagcagcagcgggcggggccgggcaaggcgggtatgggaggggcggagccgggcaaggcggggcagcagcagcggccggagcgggacaaggtgggtatggcgggcagggggctggtcaagggggggctggagcagcggccgcagcggcagccgctggaggcgcaggtcaaggtgggcaaggaggatacggtcgtggtggctacgggcagggaggtgcgggtcagggtggcgctggtacagcggcggctgcggcagcggccggaggggcgggccagggagggcagggcggatatgggcaaggtggttatgggcaaggaggagccggtcaaggaggggctgcggctgcagccgcagcagccgcgggaggagctggtcaaggcgggtacgggagggggggagcagggcaggggggtgcagccgcagcggctgccgccgcggcgggcgcaggacaaggcggatatggagggcagggggcgggccagggcggtgctggcgcagccgccgcggccgcagctgctggtggggcgggtcagggcggtcagggtggttatggcagaggcgggtacggtcagggtggagcgggccaagggggtgcaggggccgcagcagctgcggctgcagctggcggagcgtctcaaggcggtcaaggaggatacggccaaggcgactatggccaagggggtgccgggcaaggtggtgcggcggctgcagctgctgcagcaggaggtgcgggtcagggtggctatggaaggggcggcgctgggcagggaggggcagccgccgccgcaggggccgggcaaggagggtatgggggtcaaggggctggccagggaggcgcgggagctgccgctgctgcagccgctgctggtggcgcaggaaggggaggtcagggaggatacggcaggggggggtacggccaaggcggggctggtcaaggtggcgcgggagctgccgcggcggccgcggccgcgggaggtgcgggccagggtgggcaagggggatacggccaaggagggtacggacagggtgggacgggacaggggggcgcggctgccgcagcagcggcagccgctggcggtgcgggccaagggggttatggaaggggaggtgctggccagggaggcgcagcggccgcggcggcagctgcggcaggtgcaggccaaggaggatacggtggccagggcgcaggccaaggcggggctggggccgcagcggccgcagctgctgctgggggtgctgggcagggggggcaagggggctacggtcgcggtgggtacgggcaaggcggtgctggacaggggggcgcgggcgccgcggcagcggccgctgctgcaggtggggcgggtcagggcggtcagggtggctatggacaaggcgggtacggccagggtgggtatggacaaggtggcgctggccagggtggcgcggctgctgcagcagctgctgcggggggagctgggcagggagggtacggaaggggtggagccgggcaaggtggtgcggcagcggccgcgggtgccggacaaggcgggtacggggggcagggagcgggccaggggggcgcgggggccgccgcagcggcagcagccgcgggtggtgcagggcaaggtggacaaggagggtacggtagggggggttatgggcaaggtggcgctggacaaggaggggcgggcgctgctgccgcggcggctgcagcgggtggtgcgggtcaaggaggccaagggggttatggtcaaggtgggtatggtcaagggggcgcaggccaaggtggagcagctgcggctgcggcagcggcggctgggggagctggacaaggtggctacggaagaggtggggccggtcaaggaggggccgccgctgctgcagctgcagcagcaggtagtggacaaggcgggtacggtggtcaaggcgcaggacagggtggagctggggctgcagctgctgcagcagctgctggcggggcgggacaaggaggccaaggtggctatggacgtggtggctacgggcaggggggtgctgggcaggggggagctggcgctgccgcggccgcagcggcagctggtggtgcgggtcaaggtgggcagggaggctatggccaggggggttacggccagggtggttatggtcaaggtggtgcgggccaaggtggagctgccgcggcggcagcggctgcggcagctggtggagccggtcagggcggctacggacgtggcggggcgggccaaggtggtgcggcggccgccgcgggagctggacaaggcgggtacggtggacagggtgctgggcaggggggggccggcgctgcagcggcagcagctgcagccggcggagcgggtcagggtggccaagggggttatggtcgagggggctacggtcaggggggggcggggcaaggtggagctggagcggcggcagccgctgcggccgccgggggtgctgggcagggaggacagggcggttacgggcaagggggcaatgggcaagggggcgcaggtcaggggggcgcggcagccgccgcagcagccgccggcggggcggggcaggggggatacggaagggggggtgccggtcagggaggtgcagcagcggcggcagcagcagccgctggtgcagggcaagggggctatggaggccaaggcgcagggcagggaggtgccggcgcggcggctgcggcggctgctgctgggggagcagggcaaggaggccagggcggctacgggaggggaggttacggacagggcggagcgggtcaggggggtgctggcgccgctgctgcggcggctgcggcgggcggggcaagtcaaggtggacagggcgggtacgggcaaggggactacgggcaggggggagcagggcaggggggtgcagccgcagcagcggcagcggcgggcggcgccggccagggaggatatggtcgagggggcgctggacaggggggcgctgctgccgcagctggcgctgggcagggcgggtatggagggcaaggcgcggggcaaggcggggcgggagccgcagcagcggcagctgcggccggcggcgcgggccgtggcggtcaagggggatatggtaggggcgggtatggtcagggtggtgccggacaagggggtgcgggggcggcagcggccgcagcggctgctgggggcgctggccaggggggacaaggaggttatggccaggggggctatgggcaaggcggggccggacaaggcggcgcggccgcggctgcggctgctgcagcagggggagctggtcaaggaggttacgggaggggaggtgctggtcagggtggagcagccgctgcggcaggcgcaggtcaaggaggatacggaggtcagggtgctgggcaagggggggcgggagcggctgccgccgctgccgcagccggtggtgcaggacgaggaggccaagggggatacggtagaggcgggtacggccagggtggggctggccaaggcggggcgggagcagctgctgccgcagccgctgccggtggggcaggccagggcggccagggaggctatggacaaggaggttatggtcaagggggagccgggcagggcggggctgctgctgcagctgccgcggcggtggggggagcgggtcaaggaggatacgggcgaggtggcgctggacaaggaggtgccgcagcggcggccgctgctgcagcggccggcagtggacaagggggatacggcggccaaggtgctggccagggaggggcgggagctgcggcagctgccgcggctgccggtggtgccggacagggaggacaggggggttatggcggaggagggtatggccaaggtggagctgggcagggaggagcaggagcggcagcagcagccgccgcagccgggggtgctggacagggcggccaaggtggttacggtcagggtgggtacggtcagggcggagctggccaaggtggtgcggcagctgccgcagctgccgcggctggtggcgccgggcagggcggatatggaagaggtggagcagggcagggtggagcggcagccgctacaggggctggacaggggggctatggcggtcagggagcggggcaaggtggagctggggcggctgccgcggctgcagccgcgggaggtgctggacagggcgggcaaggcggatacgggagggggggttatggtcaaggtggcgcgggacaaggtggggcaggagctgcggctgctgcggctgctgctggcggagcaggtcagggtgggcagggggggtacggtcaaggtggctatgggcaaggcggggcgggtcagggcggtgctgcggccgcagccgcagcggcaggcggagctggacagggaggctacgggcgcggaggcgccggtcagggtggtgctgccgcagccgccgcagcagctgctggcgccggccagggaggttacggcggccagggcgccgggcaagggggcgcgggggcagcggcagcggcggctgccgctggtggtgccggacagggcggtcagggcggatatggtcgaggtggatatggccaaggcggggctggacagggcggagcaggggccgccgcagccggaggagccggtcaaggtggtcaggggggttatggccagggtgggtacggacaagggggagcgggccaggggggggcagcagcggccgcggcggccgcggcggccgggggagctggacagggaggttatggcggttatgggcagcagggcggagctggcgccgccgcagcagccgcttccggaccaggccaaatctattatggcccccagagcgttgcggctcccgcagcggctgcagcc

SEQ ID NO: 37 - DNA, 1293 bp, Nephila clavipes
ctaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggatccgggctaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatgggggactaggaagccaaggggcgggtcgaggtggcttaggtgggcaaggcgcgggagctgccgccgctgcagcaggtggggctggacagggtggttatggggga

SEQ ID NO: 38 - DNA, 2886 bp, Caerostris darwini
ccccaatctccatatgggccaggtccccaagggcctgggccccaaggcccagggcctcaggggccgtccggacccggcccgcagcgacctcaaggaccggggccacaagggccctatgggcctggaggtgtctctgtagtgtccgcgacggtgagcgggccagggcctcaaggcccgtccggacccggaccccaaggaccgtatggacccggcccacaaggaccgggaccacagggacccggacctcagctgcccggcccacaaggtccttcaggaccaggtccacaaggtccatatggcccaggacctcaaggaccgggaccgcagggacccggacctcaggggccatctggacccggccctcaacagcctcaaggtcccgggcctcaacgcccttacgggccggggggtgtcagcgttgtgagtacaaccgttagtggacccggaccccaagggccactaggacccggcgcgcaggttccctacggacccggtccgcaagttcccgggccacaaggcccgggaccacaaggacccagcgggcccggccctcagcgtccccaaggaccgggacctcagggaccatatggaccaggaggcgtcagtgtagttagccagacagtcagcggtccgggtcctcaaggacccagcggacctggcccccaaggtccatatggtccaggacctcaggggcctgggcctcaggggcctgccccacagggtcctagcggtcctggtccccagcgaccgcaagggccaggcccgcagcgaccgtatggaccagggggaatatcagttgtttctactaccgtatcaggtcctggaccccaggggccttccgccccgggaccgcaagggccctacggacctggtccacaggtaccaggaccgcaggggccggggccccagggacccagtgggcctggtccccaacgaccacaaggtcccggcccccagggaccgtatggccctggaggagtctctgtcgtgtcacagacagtttccggacctgggccccaaggtccgtctggccctggtcctcaagggccgtatggccccggacctcagggaccagggccacaagggccgggtccgcaagggcccagtggggcgggccctcaacgcccccagggtcctggaccacaaggaccttacggccccggcggagtttctgtcgtgtcagcgactgtgtctgggcccggtcctcaagggccgtctggcccgggtcctcaaggtccatatggtcctggaccgcagggtcctggaccgcaaaggccagtcccccaagggcccagtggtcctcgccctcagcaaccacaagggccaggaccacaacgacctttcggtccggggggcgtcagtgcagtaagcacgacagtttttgggcctggacctcaaggtccatccggtcctggtcctcaaggtccctatggtcctgggcctcaaggcccgggtccacaaggtccaggaccgcaaggaccctcgggtcaaggtcctcaacgaccctctggtccgaggccacaagggccctatggacccggtggaatttctgttgtgtcggcgacggtgagtggccctgggccacaagggccgtcgggtcccggtccacagaggccttacggtcctggaccagaaggtccaggtccgcaaggggcagggccacaaggtcccggtttacaaagacccagtgggcctggtccacagggtccttatggcccagggccgcgaggaccgccgcaagggccgtatggtcctggtccccagggacctggcccgcaaggcccaggacctcaaggaccagggccacgaggcccatccggacctgggccgcagggtccgtacggcccaggtggcgtatcagtggtctccgcttccgtgagcggcccaggtccgcaaggacctagcggacccggaccgcagggcccgtatgggccaggacctcagggtcccggaccacagggtcctggtccccagggtccagggccccagggccccggcccccgtggcccaagcggccccggaccgcagggtccgtatggtcccggcggcgtcagtgtggcctcagctagcgttagtggacctgggccccaaggtcccagtggcccgggaccgcaaggcccgtacggcccgggtccacaaggtccgggtcctagagggccgggtccgcagggtcccggcccacagggaccgggacctcagggcccatccggacccggtccacagggtccgtacggacccgggggagtcagcgtcgtatcagccagtgtatctggtccggggccacaggggccctctggtcccggaccgcaagggccatacggccccgggccccaaggccctggccctcaggggcccggcccccaagggccaggacctcaaggtcccgggccaaggggaccatctggacccggcccccaagggccgtacgggcctgggggcgtctctgttgtaagtgctagcgttagcggccctggcccgcaagggccgtctggcccagggcctcagggaccatacggtccggggcctcagggtccaggcccgcaagtgccgggtccacagggccctggcccccagggtccaggtccccaaggaccttcgggtccagggccccaaggcccctatgggcctggcggcgtctctgtagtaagtgcgagtgtttcgggtcctggaccacagggtcccagcgggcccggcccccagggaccatatggtccgggcccgcaagggcccgggccaagaggtccaggtcctcaaggacccgggccccaaggaccaggaccacaggggccctcaggtccggggccgcaaggaccgtacggacccggcggagtgagcgtggtttctgcctccgtatcgggacctggcccgcaaggtccctctggtccc

SEQ ID NO: 39 - DNA, 1000 bp, Bombyx mori
tccaaatattattcgtgtattgtttatagcctttgtcaagtcttttacaaggcaagataataagtaatattccgtgattggacgtaacatttcccggaagatccttagccgataagtcgaagagccgcatgtggctagagagacgcgggtttccgaccactggcttaggcgcttattccgccataatagatgtacgtgttcacaattagcacccgaaattcgtaatagctacgagaagtatcgaatatcaaaaatctatatattaatacgtgaagcaaaaactttgtatccctttttacgaaaattgcgaggacggaggagtatgaaatttcccacacttatagagaatacagagaagaagtgcacaatgctaatatttttttaaaataatgcataaaagatactttaaatcaataaagaaaacagcacacacactacataccatgtatttgacgcacacacgcatgtatactatttattgtcaaacttttgttcttgacgtctgtgttcaaactgagaatagattaaatattgtttgtctttattaatattttttaatagtgtagtcttggcgaaatttgtgattatagaagtataaaatacaatcataatagtgtacaaacttacaattccaattaattatagtcgaatttcgactactgcgggacctctagtattaataattctctttaaaaaaaaacagagcatcaaatactgcacaaatgtcaagcgggtctcaacgagccatgaataaattagaaatcaattaataacataaaataggcaaacaaaataaaaccatttacatagagaacgtttgttgaacaaaaacaataacttgtatacattgtttgcacaaatgtttgaagcgaaaatttattactctctacgtaagcttgatcaaacttcgttttcgtataaaacgcgttggcccaaccactttggcatagtcgtcttatcatcgggtctctaaggatcaagcgatccaaagaccgccaac

SEQ ID NO: 40 - DNA, 1000 bp, Bombyx mori
taatacaactaaacacgacttggagtattccttgtagtgtttaagattttaaatcttacttaatgacttcgaacgattttaacgataactttctctttgtttaactttaatcagcatacataaaaagccccggttttgtatcgggaagaaaaaaaatgtaattgtgttgcctagataataaacgtattatcaaagtgtgtggttttcctttaccaaagacccctttaagatgggcctaatgggcttaagtcgagtcctttccgatgtgttaaatacacatttattacactgatgcgtcgaatgtacacttttaataggatagctccactaaaaattattttatttatttaatttgttgcaccaaaactgatacattgacgaaatgatgcaatagttagcggctgacttatgcgtcgagggttgtgggttcgattcccacgtcgggcaaacattctgtaatgaacaggtttgctcttcgtctgggtgattattatctatatatatatataaatgtatataagacagtttttggtatacataaaaaagggaatcataaattggggatgaccgtccgggtgaccgtgcgttcttacttatttatttcccagatgacggacgagctcacagcccgttgaggtgtcatcgatcgagtggaatcgaaatacattcgttgcctttttattcgtttattgcttagatagatggacgagctcacggcccgcctgatgttaaggggttaccgtagctcatagacatctacaaagtaaatgccgccacccaccttgagagttctctcggttattacagtacaacggctgccccgcccttcaaactgaaatgcgttactgcttcacggcaaaaatagacctttgaggaggtgttacctactcgtggggactcacaagacgcccccaagacctggcatttacactgtgatgtctatcaactccaataatcacttaaacacgtcagctaataatgctccagtctaaacaggaaa

SEQ ID NO: 41 - DNA, 1000 bp, Bombyx mori
ggttcgcattccagtggtagattcaattcgctgctcttgctagggctagtgttagcaaattccttcgggttaagcccgagagctcacctatccgtccgcgctaagctggaaaagccccttaagctgttttttttttgtatagcctttattgctaatactaaacaataactaataattttacatacagtaacaaattgttttaacttaaatctaatacatcggatttcccggttcagtgatcagcgtgtcctgtgacacataggcctcttccagctgctttcatttttctctattggtagcttttcttgaccagattgtctctccaatcatcttgatatcgtctgtccatcttctagcttgcctggctcttttcctttaaaccaggggtcgtgaattcaatcctcacaggaagccgggattaggtgggagaatatagttccgatgttttgaatgctttatattttctgtggtcgaaaatgatactagagctacgcgtcgacaattgaatattatgctaactaccctctatttattaaaagacttttacgattcatttcgcacagaaccaatcgactgggtttagaggtttagcagtttgttgaatgaactcgttttcatcttcacgattagaggatcccaggtgttaggtaaaggatattctagattgcaggagatttttcataaataatcacgcgatggagcggtaatcagccaacatagtcgatcggcatcattattggagaccaaacaacacttcagttatccaagcgcgtcttaagtcgcattcggataatcttgaatagcctggaagtgaatttttaaaaagtttgtctcgaacaaacatcaattactttgtaattgaaccgaaaaaagaggataaacattattagcattcgttgtaatgaaatataatgttgacacagtttgaccgacgtgcactgtcttttgtggcaccggctatataaaggtggtctgtccgttctgagccacacgagtcatc

SEQ ID NO: 42 - DNA, 1000 bp, Bombyx mori
cgaatgggaacatcaactttaacaaaccttgcattaccttatggctttgtgtagttttaagtttgtgaatgcatttaaaaattgtgatggccgttcctcgtttggttgactcatttttaacagtttgtagctagttaaaataagtcgccgcgtgtatcattggctatcacacaaaaaatataactttttaaataaaaacctaagcatacatttgtattgtttcactcacggatatttactatccatgagcactgcgcatcggcaatttatgacgtataaatattcagcttgcttttaaagcttattgtttaatgtcactttagaattgggtcgtttagttttgtttggaaatagttgggattcataattcctcaggtgacagaagcaagccgtatcgccacgcattccgaaaacctcgtttgaagaaaaatatgttatagttctcgggaacaccgtggagggaaccgcccgattagcctccttaagtctctaggcaaactgttcgagcttctactctacaaaggcttcagagaattcgtctcatccaagggcattacgtgcaaactaacctaaacatcagcccgctgagtttcttgccggatcttctcagcgggtcgcgattccgatccggtagtagattcattcgcgatgtagctgcccttgagttgtttgttaggtctcctttggaaacgctcgggcagctgctagcaaatcccacccctccttgctgagcctctgctcgcccacctgttctggtgaaactggagaggcctacgggctaccagcaataccacaaacaaaaaaaaacattttggatttcgcgcaaatcactcatgcgtacaacaggtgcaccgcgttcgacaaagtttggcacaatggtttgacttacaaacttttgcggacctcttttgagtccgcacgggtgggtaccaccatcccgcctatttctgctatgaagcagtaatgcgtttctttttgaagggtgaggcagccgttgtaa

SEQ ID NO: 43 - DNA, 1000 bp, Bombyx mori
atagcttttatagcgggctttgagcgcggcgaccgaatcaagaaatttcgtaacgaaaataaacctaacacccccactccgcctaccatgtagctcgcgttcaacaccttaacacgttgcgcttgtgtagtgtttgtgaataagcgcgcggcgtgacgtcgcactgcacgcgcatcatgaaaagtctgcccatctctctctcacgcggtctagctcatgagttctttttttttattgttttaatattaaattattattgtctgattataattacataagttgattaaaaatgctatgcaatagctttaccgcagcagtccccgagggccacatgtgttttttttaaactaaacagttttaaaagttttaaatacaaaaggtttttctttacaaatatttataactttggaacctatgggtctgcggagggactttagttctctctgcattctgtacggtatgtggagagtttctaccatcgtaccgcccgccaccagagtagagttcatccatactacctggagccactgcgttcatccacagtgcgtttccagagatcttttttgccacataccatccggctttggaatgagctcccctccacggtgcttcaaacgaggcttgtggagagtacttaacggtaggttggcttggctaggcttgactctgcccctggcattgctgaagttcatggacgacggtaaccacttaccatcaggtgggccgtatgctaatccgactacaagggcaacagcaaaaaaagttaattttacaaaagtatcataattccgaggctcgctaaagatgttcgtagcatatgttacagaaacaaaaaaaaaaacaatttaaatgcgttatagaaaaacaagtgtattaaacaaataattaattattaattttattggtaactgtttattcataaagggaatatttctccaacaaattagtataaatagccggcctttgggcgtttacagacagagcaatcgaagcttcgaag

SEQ ID NO: 44 - DNA, 1000 bp, Bombyx mori
taaaacggccaacgatttatatgtttcatgttaatagcgataaaggtaatattatttgggcaccgttccgcacctcgtttaattaccatagcgggcacgatgcgatggtttctgtaacgattcgtcaattatttcgttaatagaactacatataagacttcctttatcgatattaatgtgagctgaaaacgcaaaacacaatatacgatagcttttattacatttagaacgcaaaattcatattggaagaacattaacatgtttactgtaaaactatttaacttttggatataacataaataaattacgaacattaatcgctgtttctaatttacatcccaagacaattcatcatgttacaatataaacttctaattgtaaacgtcggcgcaacgtcaccatttagttaactaaaaatagttgaaattaactgtttccttaatgttggcatgtcgactgtttaaaacggtacatattcaagagaattcatatttttttattatttttttattgcccttgtaggcagttgagcacacggcccatctgatggtgagtggttaccgtcgctcatggacttcagcaatgccaggggcagagccaagccgctgcctaccgcttaatactctccataagcctcgtttgaagaaggacatgtcatagcgctcgggaaacaccgtagaggggagctcattccatagccggatggtgcgtggcaaaaaagatctctggaagcgcactgtggatgaccgcagtggctccaggtagtatggatgaactctactccggtggtgggcggtgcggtggtaaaaacgagatgccggtatcatctcgaataattcctcagagcactccccatggaacatacggtacaaaatacagagggaaccgaagtccctccgcagacccagaggttccaaacgatccgagagaatgggattatcgacaatccgaacggccctcttctgtagggagtcaaatggaagaagctggtatttgggag

SEQ ID NO: 45 - DNA, 2970 bp, Homo sapiens
ggtccacccgggccccctggaccaccggggccacctggactgggcgggaattttgcgccccaattgtcatatggttatgacgagaagagcacgggaggaatatctgtcccagggccgatgggacccagtgggccccgtggattgccaggtcccccaggggctccaggcccccaggggttccaggggccacctggggagcccggcgagcctggagcgagtgggccgatgggaccaagaggcccacctggaccccccggaaaaaacggagacgatggcgaggcaggaaagccggggcgacccggcgagcgtggtccccccggaccccagggagctcgtgggctgcccggcactgcaggtttaccagggatgaaaggccatcgaggattttctggattagacggtgctaagggcgacgctgggcccgcaggccctaaaggggagcccggcagccctggtgagaacggagcgccagggcaaatgggtcctagaggcctaccgggtgagcgaggaagacctggcgctccggggcctgctggaaatcccggggcggacgggcaacccggtgcaaaaggtgcaaatggtgcgcccggaatagctggcgcgcctggtttcccgggggcacgtgggccgagcggaccgcagggtcctggtggtccgccgggccctaaaggaaactccggagagccaggcgcaccagggtcgaagggggataccggcgcgaagggagaacctgggccggtaggggtacaggggccacctggccccgcaggggaggaaggtaagagaggtgctagaggagagcccggtcctacaggcctccccggcccacccggtgagcgtggaggcccgggaagtagaggtttccccggtgctgacggcgttgccgggccaaagggtccggctggtgagcgagggtcacccggacccgcagggcccaaaggctcgcccggagaggccgggcgcccaggtgaggctggcctgcctggagcgaaagggctgacgggatccccggggagcccgggaccggacggaaaaacaggacccccgggaccagccggccaagacgggcgacccggccctcctgggcctcctggcgcgcgaggtcaagccggagtcatgggctttccgggtcccaagggtgccgctggagagccaggcaaagcgggcgaacgtggcgtgccaggaccaccgggtgcagttggtcctgcaggtaaagatggtgaggcaggggcacagggtccgcccggtccagctggccctgcaggtgagcgaggggagcaggggccagctgggtctcctggatttcaaggattgcccgggccagccggaccgccgggcgaagccggcaaacccggcgaacagggggtgccaggggacttaggtgctccgggaccctcgggggcgcgaggagagagaggttttccgggtgagcgaggtgttcaaggaccacccggtccagcaggcccaaggggggccaacggagcaccagggaatgatggtgcgaagggcgacgccggggccccgggtgcgccgggaagtcagggagcgccgggcctgcaaggtatgccgggagagcgtggggcggctggattgccgggtcctaagggcgatcgcggcgacgctgggccaaagggtgccgatggaagtcccggcaaagatggagtacgtgggttgactggtcccattggcccgccggggccagcgggtgcgcctggagacaagggagaatcgggtccttccgggccggcgggtcccacaggggcgagaggtgcaccgggggaccgcggcgaaccgggtccacctggcccagctgggttcgctgggccccccggggcggacggtcaaccaggagccaagggagagccaggcgacgctggcgccaagggagatgcggggccccccggacctgccgggcctgcagggccccccggacctataggaaatgttggtgcccccggggcaaaaggcgcgcgcgggtccgcgggcccgccaggggcgacgggttttcccggggccgctggaagggtgggcccacccggaccttccggaaacgccgggcctccaggaccgcctggtcctgcggggaaagaaggaggaaaagggccacgcggagaaacgggcccagcgggaagaccgggagaagttggaccacctggtcctccgggccccgctggggagaaaggaagcccaggcgctgacggaccggcgggtgcgccaggaacacccggacctcaaggcatagcaggccaaagaggagttgttgggctgccgggtcagagaggagaacgaggctttcctggcctgcccggcccttcaggcgagcccgggaaacagggcccttcaggggcatctggagaaaggggcccgccaggacctatgggtcccccggggctagccggacctccgggcgaatcaggcagggagggagcgcccggggccgaaggttcgccggggcgcgacggatcgccaggagctaaaggggaccgaggtgaaactggaccagcgggccctccgggggcaccgggtgctccgggcgccccggggcctgtggggcccgcagggaagtccggtgaccgaggtgagactggcccagcgggcccagctggaccagtcggtcctgtcggggcccgtgggcccgcaggaccccaaggccctagaggggataaaggcgaaaccggggagcagggagaccgagggatcaaaggacataggggctttagtggtctgcaagggccaccaggtccccccggttctcctggggagcagggcccctctggggccagcggcccagctggcccccgaggccccccaggatctgccggtgcaccggggaaggacggtcttaatgggttgccagggcccatcggacccccgggcccgcgtggacgcaccggagacgccggcccagtaggcccgcccggcccgcctggtccgcctggtccgccgggccccccttcggcagggtttgac

SEQ ID NO: 46 - DNA, 4194 bp, Homo sapiens
atgtttagctttgtggatctgcgcctgctgctgctgctggcggcgaccgcgctgctgacccatggccaggaagaaggccaggtggaaggccaggatgaagatattccgccgattacctgcgtgcagaacggcctgcgctatcatgatcgcgatgtgtggaaaccggaaccgtgccgcatttgcgtgtgcgataacggcaaagtgctgtgcgatgatgtgatttgcgatgaaaccaaaaactgcccgggcgcggaagtgccggaaggcgaatgctgcccggtgtgcccggatggcagcgaaagcccgaccgatcaggaaaccaccggcgtggaaggcccgaaaggcgataccggcccgcgcggcccgcgcggcccggcgggcccgccgggccgcgatggcattccgggccagccgggcctgccgggcccgccgggcccgccgggcccgccgggcccgccgggcctgggcggcaactttgcgccgcagctgagctatggctatgatgaaaaaagcaccggcggcattagcgtgccgggcccgatgggcccgagcggcccgcgcggcctgccgggcccgccgggcgcgccgggcccgcagggctttcagggcccgccgggcgaaccgggcgaaccgggcgcgagcggcccgatgggcccgcgcggcccgccgggcccgccgggcaaaaacggcgatgatggcgaagcgggcaaaccgggccgcccgggcgaacgcggcccgccgggcccgcagggcgcgcgcggcctgccgggcaccgcgggcctgccgggcatgaaaggccatcgcggctttagcggcctggatggcgcgaaaggcgatgcgggcccggcgggcccgaaaggcgaaccgggcagcccgggcgaaaacggcgcgccgggccagatgggcccgcgcggcctgccgggcgaacgcggccgcccgggcgcgccgggcccggcgggcaacccgggcgcggatggccagccgggcgcgaaaggcgcgaacggcgcgccgggcattgcgggcgcgccgggctttccgggcgcgcgcggcccgagcggcccgcagggcccgggcggcccgccgggcccgaaaggcaacagcggcgaaccgggcgcgccgggcagcaaaggcgataccggcgcgaaaggcgaaccgggcccggtgggcgtgcagggcccgccgggcccggcgggcgaagaaggcaaacgcggcgcgcgcggcgaaccgggcccgaccggcctgccgggcccgccgggcgaacgcggcggcccgggcagccgcggctttccgggcgcggatggcgtggcgggcccgaaaggcccggcgggcgaacgcggcagcccgggcccggcgggcccgaaaggcagcccgggcgaagcgggccgcccgggcgaagcgggcctgccgggcgcgaaaggcctgaccggcagcccgggcagcccgggcccggatggcaaaaccggcccgccgggcccggcgggccaggatggccgcccgggcccgccgggcccgccgggcgcgcgcggccaggcgggcgtgatgggctttccgggcccgaaaggcgcggcgggcgaaccgggcaaagcgggcgaacgcggcgtgccgggcccgccgggcgcggtgggcccggcgggcaaagatggcgaagcgggcgcgcagggcccgccgggcccggcgggcccggcgggcgaacgcggcgaacagggcccggcgggcagcccgggctttcagggcctgccgggcccggcgggcccgccgggcgaagcgggcaaaccgggcgaacagggcgtgccgggcgatctgggcgcgccgggcccgagcggcgcgcgcggcgaacgcggctttccgggcgaacgcggcgtgcagggcccgccgggcccggcgggcccgcgcggcgcgaacggcgcgccgggcaacgatggcgcgaaaggcgatgcgggcgcgccgggcgcgccgggcagccagggcgcgccgggcctgcagggcatgccgggcgaacgcggcgcggcgggcctgccgggcccgaaaggcgatcgcggcgatgcgggcccgaaaggcgcggatggcagcccgggcaaagatggcgtgcgcggcctgaccggcccgattggcccgccgggcccggcgggcgcgccgggcgataaaggcgaaagcggcccgagcggcccggcgggcccgaccggcgcgcgcggcgcgccgggcgatcgcggcgaaccgggcccgccgggcccggcgggctttgcgggcccgccgggcgcggatggccagccgggcgcgaaaggcgaaccgggcgatgcgggcgcgaaaggcgatgcgggcccgccgggcccggcgggcccggcgggcccgccgggcccgattggcaacgtgggcgcgccgggcgcgaaaggcgcgcgcggcagcgcgggcccgccgggcgcgaccggctttccgggcgcggcgggccgcgtgggcccgccgggcccgagcggcaacgcgggcccgccgggcccgccgggcccggcgggcaaagaaggcggcaaaggcccgcgcggcgaaaccggcccggcgggccgcccgggcgaagtgggcccgccgggcccgccgggcccggcgggcgaaaaaggcagcccgggcgcggatggcccggcgggcgcgccgggcaccccgggcccgcagggcattgcgggccagcgcggcgtggtgggcctgccgggccagcgcggcgaacgcggctttccgggcctgccgggcccgagcggcgaaccgggcaaacagggcccgagcggcgcgagcggcgaacgcggcccgccgggcccgatgggcccgccgggcctggcgggcccgccgggcgaaagcggccgcgaaggcgcgccgggcgcggaaggcagcccgggccgcgatggcagcccgggcgcgaaaggcgatcgcggcgaaaccggcccggcgggcccgccgggcgcgccgggcgcgccgggcgcgccgggcccggtgggcccggcgggcaaaagcggcgatcgcggcgaaaccggcccggcgggcccggcgggcccggtgggcccggtgggcgcgcgcggcccggcgggcccgcagggcccgcgcggcgataaaggcgaaaccggcgaacagggcgatcgcggcattaaaggccatcgcggctttagcggcctgcagggcccgccgggcccgccgggcagcccgggcgaacagggcccgagcggcgcgagcggcccggcgggcccgcgcggcccgccgggcagcgcgggcgcgccgggcaaagatggcctgaacggcctgccgggcccgattggcccgccgggcccgcgcggccgcaccggcgatgcgggcccggtgggcccgccgggcccgccgggcccgccgggcccgccgggcccgccgagcgcgggctttgattttagctttctgccgcagccgccgcaggaaaaagcgcatgatggcggccgctattatcgcgcggatgatgcgaacgtggtgcgcgatcgcgatctggaagtggataccaccctgaaaagcctgagccagcagattgaaaacattcgcagcccggaaggcagccgcaaaaacccggcgcgcacctgccgcgatctgaaaatgtgccatagcgattggaaaagcggcgaatattggattgatccgaaccagggctgcaacctggatgcgattaaagtgttttgcaacatggaaaccggcgaaacctgcgtgtatccgacccagccgagcgtggcgcagaaaaactggtatattagcaaaaacccgaaagataaacgccatgtgtggtttggcgaaagcatgaccgatggctttcagtttgaatatggcggccagggcagcgatccggcggatgtggcgattcagctgacctttctgcgcctgatgagcaccgaagcgagccagaacattacctatcattgcaaaaacagcgtggcgtatatggatcagcagaccggcaacctgaaaaaagcgctgctgctgcagggcagcaacgaaattgaaattcgcgcggaaggcaacagccgctttacctatagcgtgaccgtggatggctgcaccagccataccggcgcgtggggcaaaaccgtgattgaatataaaaccaccaaaaccagccgcctgccgattattgatgtggcgccgctggatgtgggcgcgccggatcaggaatttggctttgatgtgggcccggtgtgctttctg

SEQ ID NO: 47 - DNA, 1893 bp, Homo sapiens
ggtgcgggtctcggggcgctaggtggaggagctttgggcccaggtgggaagccgctaaagcctgttccaggtggccttgcaggggcaggccttggtgcgggtctcggagcctttcctgcggttacgtttccaggagcactggtgccgggaggcgttgccgatgcggcggccgcttataaagccgctaaagcgggcgctggattagggggcgttcccggcgtgggtggattaggtgtctcagcaggagctgtggtgccacagcctggtgcaggtgtgaaacccggcaaagttcccggcgttggtttaccaggtgtgtatcccggaggagtcttgcctggagcccgttttcccggagtcggagtcctcccaggtgtccctaccggcgcaggggtaaagccgaaagctcctggtgtgggtggagccttcgcaggcattcccggggtaggcccgttcggtggacctcagccgggtgtgccacttggttaccccattaaggcaccgaaactcccaggggggtacggactaccatataccacgggcaaacttccctatggatatggaccaggaggcgtagctggcgccgctggtaaagctggttatccaaccggcacaggggtcgggccccaagccgcggcagctgcggccgcaaaagcggctgcaaaattcggcgcgggagcagctggggtgcttcctggggttggtggtgcgggtgtacctggggtaccaggtgcgatacctgggatagggggcatagccggggttggcactcccgcagcggctgccgctgcagccgccgctgctaaagcagcaaaatatggcgccgcggcagggcttgtgcctggcggacctggctttggccccggggttgtaggtgttcccggtgccggggtccccggcgtcggtgtacctggagctggaataccggtcgtgccaggtgcaggcattcctggtgctgcagtacctggtgtagtatcccccgaggcagcggcgaaggctgctgccaaagcggccaagtacggcgcgcgcccaggagttggggtaggaggcatccccacctacggcgttggggcgggagggttcccgggttttggcgtgggagtaggtggaattcccggagtcgcgggtgtacccggcgttggaggtgtgcccggcgtgggaggggttcccggagtagggatttctcccgaggcacaggctgctgctgcagctaaagcagccaagtacggcgtcgggactcccgccgctgcagccgcgaaagcggcagccaaagcggcacagtttggcctagtccctggtgttggagttgcccctggggtgggcgttgccccgggagttggcgtggctcccggagttggcctcgccccgggcgtcggtgtcgcgccgggagtcggtgttgcgcccggcgtgggggtcgcccccggcattggtcccgggggagtagcggcagcagccaaatccgcagctaaggtagcggcgaaagcccaactgcgagcagcggcaggcttgggtgctgggatacccggcctgggcgtaggcgtaggcgtacctggcctgggggtaggagcgggtgtgcctggcctgggcgtcggagctggtgtacccggattcggagcagtcccaggcgctctagctgctgctaaagcagccaagtacggggcggcggtgccgggagtactaggagggttgggggcactcggcggggtaggaattccgggtggagttgttggagcggggcctgctgccgccgctgcagcggccaaagctgcagcaaaagcagcacaatttggactggttggggccgccggactcgggggactgggagttggcgggcttggagtccccggggttggtggacttggtggaatacctccggccgcggcagctaaggcggcc

SEQ ID NO: 48 - DNA, 1419 bp, Homo sapiens
atgacgacgtgttctcgtcaatttacaagcagtagctctatgaagggctcctgcggaattggaggcgggatcggtggagggtcgtcccgcatctctagtgtcctcgctggaggatcttgtagggcgccttctacttacggtggcggtctatccgtgtcatcgcgcttttctagtggcggcgcatgcggcctaggagggggctacggcggcggtttctcatccagcagcagctttggctctggtttcgggggaggctacgggggtggtttaggtgcaggcttcggtggaggactgggcgccggatttggtggcgggttcgcggggggtgatggtctcttggtcggatccgagaaagtgactatgcagaacctaaacgatcgtcttgcctcatacctcgacaaagtccgtgccttggaggaagccaatgcggatctcgaggttaaaataagggattggtatcaacgccagcgtccttctgaaataaaggattattccccgtactttaagacaatcgaagacctaagaaacaagattattgcggctactatagagaatgctcagcccatacttcagatcgacaacgcccgcctcgctgctgatgacttcagaacgaagtacgagcatgaattggccctaaggcaaacagtcgaagctgacgttaacggactgcgcagagtgttggacgagttaaccttagcacgaactgatttggagatgcaaattgagggcttaaaggaagaactagcttatttgcgtaagaatcatgaagaagagatgttagcgttacgcggccagaccggaggggatgttaacgtggaaatggatgccgcaccgggcgtagatctcagtaggatcttgaatgagatgagagaccaatacgaacaaatggccgaaaagaataggcgtgatgcggaaacatggtttctatcaaagacggaagagctgaataaggaggttgcctcgaatagcgagcttgtacaatcgagcagaagtgaggtcacggaactgcgtagagttctgcagggtttagaaattgagttgcaatcacagttgtcgatgaaggcgagtctggaaaacagcctggaggaaacgaaagggcgctactgtatgcaactctcacaaattcagggtttaatcggtagcgtagaagagcagctggcgcagctacgctgcgaaatggagcagcaatctcaggagtatcagatattattggatgtcaagactcgcctcgagcaagagatagcgacttaccgccgattgctagaaggagaagacgcgcacctctcaagtcaacaagcgagcggccagtcttactcttcacgtgaggtcttcactagcagctcttcgagctcatcgcgacaaacgcgcccgattttgaaggaacaatcatccagtagcttttcacaaggacaaagttca

SEQ ID NO: 49 - DNA, 3348 bp, Rhyacophila obliterate
tcagaatccgaatctgattcggacagcagcgaggactccgggagcaccgaaaaaattataaaacagatcataattgttcaagagaagcccaagcatggacatcatcatgccaaagagaagatctacgaagaggagattattattaagaagataggagatttacccaaaaaagactgtgatgaaaataagccgagggaagtcacctcatggcgccacactcttccgaacagaaagagtatatcgataagtaagtctattagtatagagcaaattataaagcctcacgttataacccgaataagaacatccaaaagcagcagtctaagtataagtgtcgagcgacctaggcgaattatatcacctataatttcgggaggatggaacaggcacaaaccaaggaaatattcggccagttcctccatttctaagagtattagtattgagagagttattacacctgcagtatggactaggattcacaagtcggtgagcgtcagccatagcgtgtcagttgagcaccgcaggagaatagcacctattgtggtggactactcgatctcctcatcattatcaatatcaggtgaggggcgaggattgggtagagggaagcatggttggggggggcttggccacggcggattgggtggccttgggcatggaggtttaggtggcctgggccatggtggactgggtggactcggtcacggtggcttgggtggtctcggctccgattcaggcgacctaagtatctctagtagccttagtattagcggtgagggacgaggcctcgggcgtgggaaacacggttggggtggacttggccacggcggtttaggaggattgggccacggagggttgggggggttgggccatggaggccttggtggcctgggccacggaggactgggggggctaggctcagattcgggtgacctaggcggaatcggtggcctactaggacccggagggcgccgtggctggggtcgtcgcggaccgggcaaatactcagcatcagtttcaatctcacactctatcagcatagaacgtgtgatcacacccggggtttatacaagcattcaccgatctgtttccgtaagtcactcggtgagtgttgaacataggcgcaggattgcgcctatagtagtcgactactcaatctcgtcgtcgctgtctatctcgggtgaagggcgaggcttgggacgcggtaaacatggctggggcggtttaggccatggaggacttggaggattagggcacggcggtttagggggtctaggacatggaggattggggggcttagggcatgggggacttggcggattaggctcagacagcggcgatctaggcgggattgggggccttttgggccctggggggcgcagaggttggggaaggcgaggcccaggtaaatattcggcttcggtatccattagtcattcaatttcgatagagcgagtaatcacgccgggggtatatacatccattcaccgaagtgttagcgtatcacattctgtaagcgtcgagcataggcgcagaatcgcacccatcgttgtggactattcgatatcgtccagcctctcgatatcaggcgaaggccgtggtttgggacgaggcaagcacggttggggggggttaggccacggaggcctgggtgggttaggacatggtggactcggcggcctagggcatggaggacttggcggtttgggacacggcggcctgggcggcctagggtcggattctggagacctgggcggcataggtggtttgctaggacctggtggccgcaggggctgggggagaagaggtcctggcaaatacagcgcgtccgtgtcgataagccatagcattagcatagagagggtcatcacaccaggggtctacacctcaatacacagaagtgtaagtgtgtctcactcagtttccgttgagcaccgcaggcgtattgcgcccatcgtagtagactatagtatctcgtcgtcgctatctatttccggggaggggaggggcctcggaagaggcaaacacggttgggggggtttgggtcatggcggcctaggaggtcttggacatggtggattaggaggactcgggcacggaggcctaggggggttaggacatggaggccttggaggcctgggatccgatagtggagatttaggcggaatcggaggcttgctgggccctggggggcgccgaggctggggacgcagagggcctgggaagtatagtgcgtcagtgagtatctcgcattctatatcgatagaacgagttatcactccaggagtgtatacgtcaatacaccgaagtgttagtgtctcgcattcggtatctgtcgaacacagacgccgtattgcgcctatcgtggttgattattcgattagtagctcgctatctatatccggcgaaggtcgcggcctcggtagagggaaacatgggtggggtggtctagggcacggtgggttaggaggcctcggccacggggggctaggtgggctggggcatggaggtctgggtgggttggggcatggtggtcttggtgggctcggctcggattcgggggatcttggaggcataggcggattactcggtcccggagggagaagaggctgggggaggcgcgggcccggcaaatactcggcatcagtctcaatctctcactcaatctctatcgagcgcgttattaccccgggcgtgtatacgagtatacataggagtgtgtccgtatcgcactcagtgtctgtggaacacagacgacgaatcgcacctatagtcgttgactacagtatctccagttcattatcgatttccggggagggacgaggcttgggacgagggaagcacggatggggcggacttggtcatggtgggctagggggtctcgggcatgggggattgggtggattgggacatggtggcttagggggactcggccacggggggttggggggcctcgggtctgatagtggcgatttagggggaatcggtggtctcctaggccccggtgggcgaaggggctggggacgccgcgggcctgggaaatactctgcttctgtttccatctctcattcgatatctatcgaacgtgtaatcacgcccggagtgtatacatctatacatagaagtgtcagcgtcagccatagtgtaagtatagaaagaccaaggaggattatctctccggttgttgattattctctatcaattagtgccagttcttcagtttccgctgaagggggtcgccgtcgtcgtttacccggttcgtgcggcactatcataagtgacagctcgagtaacgttggcggtctggggcgtataggggcgttacacaaattgggcggtgggcgcatt